SEQUENCE LISTING (25 sequences)

SEQ ID NO: 1 - protein, 166 aa, homo sapiens
MSGLGRSRRGGRSRVDQEERFPQGLWTGVAMRSCPEEQYWDPLLGTCMSCKTICNHQSQRTCAAFCRSLSCRKEQGKFYDHLLRDCISCASICGQHPKQCAYFCENKLRSPVNLPPELRRQRSGEVENNSDNSGRYQGLGHRGSEASPALPGLKLSANQVALVYST

SEQ ID NO: 2 - protein, 232 aa, homo sapiens
EPKSSDKTHTCPPCPAPEAEGAPSVFLFPPKPKDTLMISRTPEVTCVVVDVSHEDPEVKFNWYVDGVEVHNAKTKPREEQYNSTYRVVSVLTVLHQDWLNGKEYKCKVSNKALPSSIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 3 - protein, 313 aa, homo sapiens
AMRSCPEEQYWDPLLGTCMSCKTICNHQSQRTCAAFCRSLSCRKEQGKFYDHLLRDCISCASICGQHPKQCAYFCENKLRSEPKSSDKTHTCPPCPAPEAEGAPSVFLFPPKPKDTLMISRTPEVTCVVVDVSHEDPEVKFNWYVDGVEVHNAKTKPREEQYNSTYRVVSVLTVLHQDWLNGKEYKCKVSNKALPSSIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 4 - DNA, 762 bp, homo sapiens
  CDS (7)..(759)
ggatccatgaagcacctgtggttcttcctcctgctggtggcggctcccagatgggtcctgtccgagcccaaatcttgtgacaaaactcacacatgcccaccgtgcccagcacctgaactcctggggggaccgtcagtcttcctcttccccccaaaacccaaggacaccctcatgatctcccggacccctgaggtcacatgcgtggtggtggacgtgagccacgaagaccctgaggtcaagttcaactggtacgtggacggcgtggaggtgcataatgccaagacaaagccgcgggaggagcagtacaacagcacgtaccgtgtggtcagcgtcctcaccgtcctgcaccaggactggctgaatggcaaggagtacaagtgcaaggtctccaacaaagccctcccagcccccatcgagaaaaccatctccaaagccaaagggcagccccgagaaccacaggtgtacaccctgcccccatcccgggatgagctgaccaagaaccaggtcagcctgacctgcctggtcaaaggcttctatcccagcgacatcgccgtggagtgggagagcaatgggcagccggagaacaactacaagaccacgcctcccgtgctggactccgacggctccttcttcctctacagcaagctcaccgtggacaagagcaggtggcagcaggggaacgtcttctcatgctccgtgatgcatgaggctctgcacaaccactacacgcagaagagcctctccctgtctccgggtaaatga

SEQ ID NO: 5 - protein, 251 aa, homo sapiens
MKHLWFFLLLVAAPRWVLSEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 6 - protein, 293 aa, homo sapiens
MSGLGRSRRGGRSRVDQEERFPQGLWTGVAMRSCPEEQYWDPLLGTCMSCKTICNHQSQRTCAAFCRSLSCRKEQGKFYDHLLRDCISCASICGQHPKQCAYFCENKLRSPVNLPPELRRQRSGEVENNSDNSGRYQGLEHRGSEASPALPGLKLSADQVALVYSTLGLCLCAVLCCFLVAVACFLKKRGDPCSCQPRSRPRQSPAKSSQDHAMEAGSPVSTSPEPVETCSFCFPECRAPTQESAVTPGTPDPTCAGRWGCHTRTTVLQPCPHIPDSGLGIVCVPAQEGGPGA

SEQ ID NO: 7 - DNA, 1214 bp, homo sapiens
  CDS (17)..(1192)
tattaggccggccaccatggatgcaatgaagagagggctctgctgtgtgctgctgctgtgtggcgccgtcttcgtttcgctcagccaggaaatccatgccgagttgagacgcttccgtagagctatgagatcctgccccgaagagcagtactgggatcctctgctgggtacctgcatgtcctgcaaaaccatttgcaaccatcagagccagcgcacctgtgcagccttctgcaggtcactcagctgccgcaaggagcaaggcaagttctatgaccatctcctgagggactgcatcagctgtgcctccatctgtggacagcaccctaagcaatgtgcatacttctgtgagaacaagctcaggagcccagtgaaccttccaccagagctcaggagacagcggagtggagaagttgaaaacaattcagacaactcgggaaggtaccaaggattggagcacagaggctcagaagcaagtccagctctcccaggtctcaaggagcccaaatcttcagacaaaactcacacatgcccaccgtgcccagcacctgaagccgagggggcaccgtcagtcttcctcttccccccaaaacccaaggacaccctcatgatctcccggacccctgaggtcacatgcgtggtggtggacgtgagccacgaagaccctgaggtcaagttcaactggtacgtggacggcgtggaggtgcataatgccaagacaaagccgcgggaggagcagtacaacagcacgtaccgtgtggtcagcgtcctcaccgtcctgcaccaggactggctgaatggcaaggagtacaagtgcaaggtctccaacaaagccctcccatcctccatcgagaaaaccatctccaaagccaaagggcagccccgagaaccacaggtgtacaccctgcccccatcccgggatgagctgaccaagaaccaggtcagcctgacctgcctggtcaaaggcttctatcccagcgacatcgccgtggagtgggagagcaatgggcagccggagaacaactacaagaccacgcctcccgtgctggactccgacggctccttcttcctctacagcaagctcaccgtggacaagagcaggtggcagcaggggaacgtcttctcatgctccgtgatgcatgaggctctgcacaaccactacacgcagaagagcctctccctgtctccgggtaaataatctagaggcgcgccaatta

SEQ ID NO: 8 - protein, 392 aa, homo sapiens
MDAMKRGLCCVLLLCGAVFVSLSQEIHAELRRFRRAMRSCPEEQYWDPLLGTCMSCKTICNHQSQRTCAAFCRSLSCRKEQGKFYDHLLRDCISCASICGQHPKQCAYFCENKLRSPVNLPPELRRQRSGEVENNSDNSGRYQGLEHRGSEASPALPGLKEPKSSDKTHTCPPCPAPEAEGAPSVFLFPPKPKDTLMISRTPEVTCVVVDVSHEDPEVKFNWYVDGVEVHNAKTKPREEQYNSTYRVVSVLTVLHQDWLNGKEYKCKVSNKALPSSIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 9 - DNA, 1070 bp, homo sapiens
  CDS (17)..(1048)
tattaggccggccaccatggatgcaatgaagagagggctctgctgtgtgctgctgctgtgtggcgccgtcttcgtttcgctcagccaggaaatccatgccgagttgagacgcttccgtagagctatgagatcctgccccgaagagcagtactgggatcctctgctgggtacctgcatgtcctgcaaaaccatttgcaaccatcagagccagcgcacctgtgcagccttctgcaggtcactcagctgccgcaaggagcaaggcaagttctatgaccatctcctgagggactgcatcagctgtgcctccatctgtggacagcaccctaagcaatgtgcatacttctgtgagaacgagcccaaatcttcagacaaaactcacacatgcccaccgtgcccagcacctgaagccgagggggcaccgtcagtcttcctcttccccccaaaacccaaggacaccctcatgatctcccggacccctgaggtcacatgcgtggtggtggacgtgagccacgaagaccctgaggtcaagttcaactggtacgtggacggcgtggaggtgcataatgccaagacaaagccgcgggaggagcagtacaacagcacgtaccgtgtggtcagcgtcctcaccgtcctgcaccaggactggctgaatggcaaggagtacaagtgcaaggtctccaacaaagccctcccatcctccatcgagaaaaccatctccaaagccaaagggcagccccgagaaccacaggtgtacaccctgcccccatcccgggatgagctgaccaagaaccaggtcagcctgacctgcctggtcaaaggcttctatcccagcgacatcgccgtggagtgggagagcaatgggcagccggagaacaactacaagaccacgcctcccgtgctggactccgacggctccttcttcctctacagcaagctcaccgtggacaagagcaggtggcagcaggggaacgtcttctcatgctccgtgatgcatgaggctctgcacaaccactacacgcagaagagcctctccctgtctccgggtaaataatctagaggcgcgccaatta

SEQ ID NO: 10 - protein, 344 aa, homo sapiens
MDAMKRGLCCVLLLCGAVFVSLSQEIHAELRRFRRAMRSCPEEQYWDPLLGTCMSCKTICNHQSQRTCAAFCRSLSCRKEQGKFYDHLLRDCISCASICGQHPKQCAYFCENEPKSSDKTHTCPPCPAPEAEGAPSVFLFPPKPKDTLMISRTPEVTCVVVDVSHEDPEVKFNWYVDGVEVHNAKTKPREEQYNSTYRVVSVLTVLHQDWLNGKEYKCKVSNKALPSSIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 11 - DNA, 1082 bp, homo sapiens
  CDS (17)..(1060)
tattaggccggccaccatggatgcaatgaagagagggctctgctgtgtgctgctgctgtgtggcgccgtcttcgtttcgctcagccaggaaatccatgccgagttgagacgcttccgtagagctatgagatcctgccccgaagagcagtactgggatcctctgctgggtacctgcatgtcctgcaaaaccatttgcaaccatcagagccagcgcacctgtgcagccttctgcaggtcactcagctgccgcaaggagcaaggcaagttctatgaccatctcctgagggactgcatcagctgtgcctccatctgtggacagcaccctaagcaatgtgcatacttctgtgagaacaagctcaggagcgagcccaaatcttcagacaaaactcacacatgcccaccgtgcccagcacctgaagccgagggggcaccgtcagtcttcctcttccccccaaaacccaaggacaccctcatgatctcccggacccctgaggtcacatgcgtggtggtggacgtgagccacgaagaccctgaggtcaagttcaactggtacgtggacggcgtggaggtgcataatgccaagacaaagccgcgggaggagcagtacaacagcacgtaccgtgtggtcagcgtcctcaccgtcctgcaccaggactggctgaatggcaaggagtacaagtgcaaggtctccaacaaagccctcccatcctccatcgagaaaaccatctccaaagccaaagggcagccccgagaaccacaggtgtacaccctgcccccatcccgggatgagctgaccaagaaccaggtcagcctgacctgcctggtcaaaggcttctatcccagcgacatcgccgtggagtgggagagcaatgggcagccggagaacaactacaagaccacgcctcccgtgctggactccgacggctccttcttcctctacagcaagctcaccgtggacaagagcaggtggcagcaggggaacgtcttctcatgctccgtgatgcatgaggctctgcacaaccactacacgcagaagagcctctccctgtctccgggtaaataatctagaggcgcgccaatta

SEQ ID NO: 12 - protein, 348 aa, homo sapiens
MDAMKRGLCCVLLLCGAVFVSLSQEIHAELRRFRRAMRSCPEEQYWDPLLGTCMSCKTICNHQSQRTCAAFCRSLSCRKEQGKFYDHLLRDCISCASICGQHPKQCAYFCENKLRSEPKSSDKTHTCPPCPAPEAEGAPSVFLFPPKPKDTLMISRTPEVTCVVVDVSHEDPEVKFNWYVDGVEVHNAKTKPREEQYNSTYRVVSVLTVLHQDWLNGKEYKCKVSNKALPSSIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 13 - DNA, 1109 bp, homo sapiens
  CDS (17)..(1090)
tattaggccggccaccatggatgcaatgaagagagggctctgctgtgtgctgctgctgtgtggcgccgtcttcgtttcgctcagccaggaaatccatgccgagttgagacgcttccgtagagctatgagatcctgccccgaagagcagtactgggatcctctgctgggtacctgcatgtcctgcaaaaccatttgcaaccatcagagccagcgcacctgtgcagccttctgcaggtcactcagctgccgcaaggagcaaggcaagttctatgaccatctcctgagggactgcatcagctgtgcctccatctgtggacagcaccctaagcaatgtgcatacttctgtgagaacaagctcaggagcccagtgaaccttccaccagagctcagggagcccaaatcttcagacaaaactcacacatgcccaccgtgcccagcacctgaagccgagggggcaccgtcagtcttcctcttccccccaaaacccaaggacaccctcatgatctcccggacccctgaggtcacatgcgtggtggtggacgtgagccacgaagaccctgaggtcaagttcaactggtacgtggacggcgtggaggtgcataatgccaagacaaagccgcgggaggagcagtacaacagcacgtaccgtgtggtcagcgtcctcaccgtcctgcaccaggactggctgaatggcaaggagtacaagtgcaaggtctccaacaaagccctcccatcctccatcgagaaaaccatctccaaagccaaagggcagccccgagaaccacaggtgtacaccctgcccccatcccgggatgagctgaccaagaaccaggtcagcctgacctgcctggtcaaaggcttctatcccagcgacatcgccgtggagtgggagagcaatgggcagccggagaacaactacaagaccacgcctcccgtgctggactccgacggctccttcttcctctacagcaagctcaccgtggacaagagcaggtggcagcaggggaacgtcttctcatgctccgtgatgcatgaggctctgcacaaccactacacgcagaagagcctctccctgtctccgggtaaataatctagaggcgcgccaatta

SEQ ID NO: 14 - protein, 357 aa, homo sapiens
MDAMKRGLCCVLLLCGAVFVSLSQEIHAELRRFRRAMRSCPEEQYWDPLLGTCMSCKTICNHQSQRTCAAFCRSLSCRKEQGKFYDHLLRDCISCASICGQHPKQCAYFCENKLRSPVNLPPELREPKSSDKTHTCPPCPAPEAEGAPSVFLFPPKPKDTLMISRTPEVTCVVVDVSHEDPEVKFNWYVDGVEVHNAKTKPREEQYNSTYRVVSVLTVLHQDWLNGKEYKCKVSNKALPSSIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 15 - DNA, 29 bp, artificial sequence
  primer sequence
tattaggccggccaccatggatgcaatga

SEQ ID NO: 16 - DNA, 29 bp, artificial sequence
  primer sequence
tgaagatttgggctccttgagacctggga

SEQ ID NO: 17 - DNA, 29 bp, artificial sequence
  primer sequence
tcccaggtctcaaggagcccaaatcttca

SEQ ID NO: 18 - DNA, 36 bp, artificial sequence
  primer sequence
taattggcgcgcctctagattatttacccggagaca

SEQ ID NO: 19 - DNA, 30 bp, artificial sequence
  primer sequence
tgaagatttgggctcgttctcacagaagta

SEQ ID NO: 20 - DNA, 31 bp, artificial sequence
  primer sequence
atacttctgtgagaacgagcccaaatcttca

SEQ ID NO: 21 - DNA, 30 bp, artificial sequence
  primer sequence
tttgggctcgctcctgagcttgttctcaca

SEQ ID NO: 22 - DNA, 28 bp, artificial sequence
  primer sequence
ctcaggagcgagcccaaatcttcagaca

SEQ ID NO: 23 - DNA, 26 bp, artificial sequence
  primer sequence
tttgggctccctgagctctggtggaa

SEQ ID NO: 24 - DNA, 28 bp, artificial sequence
  primer sequence
gagctcagggagcccaaatcttcagaca

SEQ ID NO: 25 - protein, 35 aa, artificial sequence
  modified signal peptide sequence
MDAMKRGLCCVLLLCGAVFVSLSQEIHAELRRFRR

(19) United States
(12) Patent Application Publication
(10) Pub. No.: US 2010/0239580 A1
(43) Pub. Date: Sep. 23, 2010

(54) TACI-IMMUNOGLOBULIN FUSION PROTEINS FOR TREATMENT OF OPTIC NEURITIS

(75) Inventors: Alessandra Del Rio, Roma (IT); Gianluca Rinaldi, Monterotondo (IT); Joel Richard, Montfort L'Amaury (FR); Thomas Plitz, Geneva (CH)

Correspondence Address: ALSTON & BIRD LLP, BANK OF AMERICA PLAZA, 101 SOUTH TRYON STREET, SUITE 4000, CHARLOTTE, NC, 28280-4000, US

(73) Assignee: Area Trading S.A., Aubonne (CH)

(21) Appl. No.: 12/740,879

(22) PCT Filed: Nov. 10, 2008
(86) PCT No.: PCT/EP08/65252

§ 371 Date: Apr. 30, 2010

Related U.S. Application Data
(60) Provisional application No. 61/002,988, filed on Nov. 14, 2007.

(30) Foreign Application Priority Data
Nov. 12, 2007 (EP) 07120490.3

(51) Int. Cl. A61K 39/395; C07K 16/00; A61P 27/02; A61P 25/00

(52) U.S. Cl. 424/134.1; 530/387.3

(57) ABSTRACT

The invention relates to TACI-Immunoglobulin fusion proteins for the treatment of optic neuritis.

### FIELD OF INVENTION

The present invention is in the field of optic neuritis. More specifically, it relates to the use of TACI-immunoglobulin (Ig) fusion proteins for the treatment of optic neuritis, in particular optic neuritis as clinically isolated syndrome.

### BACKGROUND OF THE INVENTION

### The BLyS Ligand/Receptor Family

Three receptors, TACI (transmembrane activator and CAML-interactor), BCMA (B-cell maturation antigen) and BAFF-R (receptor for B-cell activating factor), have been identified that have unique binding affinities for the two growth factors BLyS (B-lymphocyte stimulator) and APRIL (a proliferation-inducing ligand) (Marsters et al. 2000; Thompson et al. 2001).

TACI and BCMA bind both BLyS and APRIL, while BAFF-R appears capable of binding only BLyS with high affinity (Marsters et al., 2000; Thompson et al. 2001). As a result, BLyS is able to signal through all three receptors, while APRIL only appears capable of signaling through TACI and BCMA. In addition, circulating heterotrimeric complexes of BLyS and APRIL (groupings of three protein subunits, containing one or two copies each of BLyS and APRIL subunits) have been identified in serum samples taken from patients with systemic immune-based rheumatic diseases, and have been shown to induce B-cell proliferation in vitro (Roschke et al., 2002).

BLyS and APRIL are potent stimulators of B-cell maturation, proliferation and survival (Moore et al., 1999; Schneider et al., 1999; Do et al., 2000). BLyS and APRIL may be necessary for persistence of autoimmune diseases, especially those involving B-cells. Transgenic mice engineered to express high levels of BLyS exhibit immune cell disorders and display symptoms similar to those seen in patients with Systemic Lupus Erythematosus (Gross et al. 2000; Mackay et al. 1999). Similarly, increased levels of BLyS/APRIL have been measured in serum samples taken from Systemic Lupus Erythematosus patients and other patients with various autoimmune diseases like Rheumatoid Arthritis (Roschke 2002; Cheema et al. 2001; Groom et al. 2002), extending the association of BLyS and/or APRIL and B-cell mediated diseases from animal models to humans. The expression of BLyS and APRIL are upregulated in peripheral blood monocytes and T cells of MS patients (Thangarajh et al., 2004; Thangarajh et al., 2005). In MS lesions, BLyS expression was found strongly upregulated on astrocytes localized close to immune cells expressing BAFF-R (Krumbholz et al., 2005).

### Atacicept

Atacicept (INN) is a recombinant fusion protein containing the extracellular, ligand-binding portion of the receptor TACI (Transmembrane activator and calcium modulator and cyclophilin-ligand (CAML)-interactor) and the modified Fc portion of human IgG. Atacicept acts as an antagonist to BLyS (B-lymphocyte stimulator) and APRIL (A proliferation-inducing ligand), both members of the tumor necrosis factor (TNF) superfamily. BLyS and APRIL have been shown to be important regulators of B cell maturation function and survival.

Atacicept is a soluble glycoprotein containing 313 amino acids, resulting from the fusion of human IgG₁-Fc and the extracellular domain of the BLyS receptor TACI, with a predicted mass of 35.4 kilodalton (kDa). The product conformation is dimeric, with a predicted mass of 73.4 kDa. Atacicept is produced in Chinese Hamster Ovary (CHO) cells by recombinant technology.

In atacicept, the human IgG₁-Fc was modified to reduce Fc binding to the C1q component of complement and the interaction with antibody receptors (Tao et al., 1993; Canfield et al., 1991). Atacicept was tested and confirmed for reduction of these Fc effector functions.

### Optic Neuritis and Multiple Sclerosis

Optic neuritis (ON) is defined as inflammation of the optic nerve. It is one of the causes of acute loss of vision associated with pain. The diagnosis of ON is usually made clinically. The classic clinical presentation of ON consists of (a) loss of vision, (b) eye pain, and (c) dyschromatopsia, which refers to the impairment of accurate color vision. Seventy percent of cases in adults are unilateral (Optic Neuritis Study Group, 1991).

The inflammation of the optic nerve causes demyelination and can be idiopathic and isolated. However, this disease has a very strong association with multiple sclerosis (MS). About 20% of cases of MS manifest as ON, and 38-50% of patients with MS develop ON at some point during the course of their disease (Chen and Gordon, 2005). According to one of the long-term follow up studies of optic neuritis, the Optic Neuritis Treatment Trial, 28% and 35% of patients develop recurrence within 5 and 10 years, respectively (Beck et al., 2003). Not surprisingly, recurrence was more common in patients who were subsequently diagnosed with MS.

The incidence of demyelinating ON is approximately 5 cases per 100′000 persons-years (Rodriguez et al., 1995), closely following the incidence of MS with approximately 4.2 per 100,000 (Hirtz et al., 2007). Like in MS, patients with ON typically present in the third and fourth decade of life; the mean age in Optic Neuritis Treatment Trial was 32 years. Women are affected more than men (Optic Neuritis Study Group, 1991).

Multiple sclerosis (MS) is a chronic, inflammatory, demyelinating disease of the central nervous system (CNS) and is one of the most common causes of neurological disability in young adults. It is characterized by multifocal recurrent attacks (relapses) of neurological symptoms and signs with variable recovery. Eventually, the majority of subjects develop a progressive clinical course.

Approximately one and a half million adults are affected worldwide. The disease is twice as prevalent in women as in men, causes considerable disability over time and continues for the lifetime of the patient.

The exact cause of MS is unknown, although an autoimmune process has been implicated. It appears that genetic susceptibility may very well play a role in disease initiation, but currently unidentified environmental factors are also likely involved. It is assumed that T cells autoreactive to CNS antigens are stimulated in the peripheral circulation and recruited into the CNS. Upon restimulation by antigen presenting cells, autoreactive T cells proliferate, and initiate a pro-inflammatory cascade within the brain. The inflammation results over time in demyelination and ultimately loss of axons and brain volume.

The paradigm of MS being mainly a T cell mediated disease has shifted during recent years (Klawiter et al., 2007). There is a common understanding in the medical community that B cells contribute to ON and MS pathology by mainly two mechanisms: 1) on a cellular level by serving as antigen presenting cells that restimulate CD4 T cells and produce pro-inflammatory cytokines, and 2) on the level of humoral immunity by producing antibody directed against CNS components. Histopathological analysis suggests B cell and antibody mediated pathology in a significant proportion of the ON and MS population. Abnormal intrathecal immunoglobulin G (IgG) synthesis, reflected as the presence of oligoclonal bands in the cerebrospinal fluid (CSF), is found in 60-70% of patients with isolated ON, suggesting an immunologic pathophysiology similar to MS. Histopathological analysis suggests B cell and antibody mediated pathology in a significant proportion of the MS population. This is corroborated by reports on the use of Rituximab in MS and neuromyelitis optica. It is therefore warranted to explore agents targeting B cell immunity as MS therapy.

Current disease modifying treatments for MS, i.e. medications modifying the course of MS, modulate or suppress the immune system. There are FDA approved immunomodulating agents for relapsing MS: three beta interferons (Rebif®—Merck Serono; Betaseron®—Berlex; Avonex®—Biogen;) and Glatiramer Acetate (Copaxone®—Teva). The FDA also approved natalizumab (Tysabri®—Biogen and Elan) under a special restricted distribution program as monotherapy for relapsing multiple sclerosis. Additionally, there is one FDA approved immunosuppressing drug for advanced or chronic MS, Mitoxantrone (Novantrone®—Merck Serono).

Several other immunosuppressive agents are being evaluated, although not FDA approved yet, such as e.g. Cladribine, a chlorinated purine analogue 2-chloro-2′deoxyadenosine (2-CdA), in the treatment of MS (EP 626 853).

Since optic neuritis is a severe disease, and since optic neuritis frequently converts to multiple sclerosis, it would be beneficial to have new and efficient possibilities to treat optic neuritis and prevent development into MS.

### SUMMARY OF THE INVENTION

The present invention is based on a clinical trial assessing the beneficial effect of atacicept in patients suffering from optic neuritis as clinically isolated syndrome (CIS).

Therefore, the invention relates to a TACI-Ig fusion protein for treatment of optic neuritis and to a method of treating optic neuritis comprising administering to a patient a composition comprising a TACI-Ig fusion protein in an amount effective to treat optic neuritis.

In an embodiment of the invention, the TACI-Ig fusion protein is for treatment of optic neuritis as a clinically isolated syndrome.

In another embodiment, the TACI-Ig fusion protein is for prevention of conversion of optic neuritis to relapsing multiple sclerosis (RMS) or clinically defined multiple sclerosis (CDMS) and for prevention of the recurrence of optic neuritis.

In accordance with the present invention, the TACI-Ig fusion protein comprises
a) the TACI extracellular domain or a fragment or variant thereof which binds to BLyS and/or APRIL; and
b) a human immunoglobulin-constant domain.

### BRIEF DESCRIPTION OF THE DRAWINGS

FIG. 1: Trial design of the two-arm randomized, double-blind, placebo-controlled, multicenter Phase II study described in Example 1.

### DETAILED DESCRIPTION OF THE INVENTION

The present invention is based on the finding that optic neuritis can be treated by administration of an effective amount of atacicept.

Therefore, the invention relates to a TACI-Ig fusion protein for treatment of optic neuritis, and to a method of treating optic neuritis comprising administering to a patient a composition comprising a TACI-Ig fusion protein in an amount effective to treat optic neuritis.

The diagnosis of optic neuritis can be made clinically by assessment of (a) loss of vision; (b) eye pain; and (c) dyschromatopsia (impairment of accurate color vision).

In one embodiment of the invention, the TACI-Ig is for treatment of optic neuritis as a clinically isolated syndrome (CIS).

A clinically isolated syndrome (CIS) is the first neurologic episode or first clinical event, lasting at least 24 hours, which is caused by inflammation and/or demyelination in one or more sites in the central nervous system (CNS). A person with CIS can have a single neurologic sign or symptom, such as an attack of optic neuritis, which is caused by a single lesion. In this case, the CIS is monofocal. A person with CIS can also have one or more than one sign or symptom, e.g. an attack of optic neuritis accompanied by weakness on one side, caused by lesions in more than one place. In this case, the CIS is referred to as multifocal.

In accordance with the present invention, TACI-Ig fusion protein is used for treatment of optic neuritis as multifocal, or preferably as monofocal clinically isolated syndrome.

Optic neuritis can affect both eyes or a single eye. In an embodiment, the optic neuritis to be treated in accordance with the invention affects one eye, i.e. it is symptomatic unilateral optic neuritis. Symptomatic optic neuritis is defined by loss of vision (e.g. a blurred vision), eye pain and dyschromatopsia.

Patients suffering from optic neuritis frequently develop recurrence (i.e. any optic neuritis event after a first clinical event of optic neuritis, such as e.g. a second attack of optic neuritis in the same eye). Therefore, an embodiment of the invention relates to the prevention of recurrence of optic neuritis by use of a TACI-Ig fusion protein.

Individuals who experience optic neuritis as clinically isolated syndrome may or may not develop multiple sclerosis.

In a further embodiment of the invention, the TACI-Ig fusion protein is used for prevention of the conversion of optic neuritis to relapsing multiple sclerosis (RMS) or clinically definite multiple sclerosis (CMDS), i.e. of development of RMS or CMDS after optic neuritis as CIS.

Relapsing multiple sclerosis (RMS) can e.g. be relapsing-remitting multiple sclerosis (RRMS), secondary progressive multiple sclerosis (SPMS) with superimposed relapses or progressing-relapsing multiple sclerosis (PRMS).

Clinically definite multiple sclerosis is established when the patient experiences a second neurologic (demyelinating) event.

Diagnosis of RMS is carried out according to the revised McDonald criteria as described e.g. in Polman et al., 2005, or in Appendix A of Example 1 below.

In relapsing MS, clinical relapses must be separated by at least one month.

A clinical attack or relapse is defined by the following three criteria (see also Appendix C of Example 1):

(1) Neurological abnormality, either newly appearing or re-appearing, with abnormality specified by both (i) Neurological abnormality separated by at least 30 days from onset of a preceding clinical event, and (ii) Neurological abnormality lasting for at least 24 hours;
(2) Absence of fever or known infection (fever with temperature (measured axillary, orally or intrauriculary)>37.5° C./99.5° F.);
(3) Objective neurological impairment, correlating with the subject's reported symptoms, defined as either i) Increase in at least one of the functional systems of the EDSS, or ii) increase of the total EDSS score.

The Magnetic Resonance Imaging (MRI) criterion for dissemination of lesions in time has been defined as at least one new T2 lesion occurring at any time point after a so-called reference scan performed at least 30 days after the onset of initial clinical event. Alternatively, an MRI scan is done at least three months after onset of symptoms and dissemination in time is established by at least one new gadolinium (Gd)-enhancing lesion. A “new” lesion is a lesion not occurring at the site implicated by the initial clinical event.

Brain abnormalities and dissemination in space are demonstrated by MRI if three of the following criteria are fulfilled: (1) At least one gadolinium-enhancing lesion or nine T2 hyperintense lesions if there is no gadolinium-enhancing lesion; (2) at least one infratentorial lesion; (3) at least one juxtacortical lesion; (4) at least three periventricular lesions. A spinal cord lesion can be considered equivalent to a brain infratentorial lesion. An enhancing spinal cord lesion is considered to be equivalent to an enhancing brain lesion, and individual spinal cord lesions can contribute together with individual brain lesions to reach the required number of T2 lesions.

The term “treatment” within the context of this invention refers to any beneficial effect on the disease, including attenuation, reduction, decrease, diminishing or alleviation of the pathological development or one or more symptoms developed by the patient after onset or diagnosis of the disease, also including the slowing-down of the progress of the disease, or of a symptom thereof.

The term “prevention” within the context of this invention refers not only to a complete prevention of a certain effect, but also to any partial or substantial prevention, attenuation, reduction, decrease, diminishing or postponement of an effect or symptom before or at early onset of the disease.

Treatment of optic neuritis in accordance with the present invention is characterized by at least one of the following (a) preservation of the Retinal Nerve Fiber Layer (RNFL) thickness as assessed by Optical Coherence Tomography (OCT); (b) preservation of visual outcomes such as low contrast letter acuity and contrast sensitivity; (c) preservation of color vision, visual field and high contrast sensitivity.

Furthermore, the following parameters can be used to assess the effects of treatment with the TACI-Ig fusion protein in accordance with the present invention:
Preservation or reduction of no more than 5% or 10% 15% or 20% or no more than 5 μm or 10 μm or 15 μm or 20 μm of RNFL thickness between the affected eye and fellow eye;
Preservation or reduction of no more than 5% or 10% 15% or 20% or no more than 5 μm or 10 μm or 15 μm or 20 μm of RNFL thickness in the affected eye over time;
Preservation or reduction of no more than 5% or 10% 15% or 20% in macular thickness at 3 mm around fovea in the affected eye over time of treatment;
Preservation or reduction of no more than 5% or 10% 15% or 20% of macular thickness at 6 mm around fovea in the affected eye over time of treatment;
Preservation or reduction of no more than 5% or 10% 15% or 20% of macular volume in the affected eye over time of treatment;
Preservation of low contrast letter acuity (Sloan charts, e.g. described by Trip et al., 2005) over time of treatment;

Preservation of contrast sensitivity (Pelli-Robson charts, e.g. described by Fisher et al., 2006) over time of treatment;

No conversion to RMS as per McDonald criteria or CDMS (second clinical attack) for at least 3 or 6 or 9 months;
Preservation of high contrast letter acuity (Early Treatment Diabetic Retinopathy Study (ETDRS) chart (measurable e.g. in accordance with the disclosure of U.S. Pat. No. 5,078,486);
Preservation of visual field (Humphrey automated perimetry (measurable e.g. in accordance with Trope and Britton, 1987); and
Preservation of color vision (Farnsworth Munsell D15 test (Farnsworth-Munsell Dichotomous D-15 Test e.g. available from www.munsell.eu).

The EDSS is a classification scheme (Rating Scale) that describes disease severity and is used to define the disease stages accepted to be enrolled into clinical trials. It is also used by neurologists to follow the progression of Multiple Sclerosis disability and evaluate treatment results, for similar groupings of people. The Functional System (FS) scale is incorporated within its overall framework.

EDSS rates are defined as follows (Kurtzke, Neurology, 1983, 33:1444-52):
0.0—Normal Neurological Exam;
1.0—No disability, minimal signs on 1 FS;
1.5—No disability minimal signs on 2 of 7 FS;
2.0—Minimal disability in 1 of 7 FS;
2.5—Minimal disability in 2 FS;
3.0—Moderate disability in 1 FS; or mild disability in 3-4 FS, though fully ambulatory;
3.5—Fully ambulatory but with moderate disability in 1 FS and mild disability in 1 or 2 FS; or moderate disability in 2 FS; or mild disability in 5 FS;
4.0—Fully ambulatory without aid, up and about 12 hrs a day despite relatively severe disability. Able to walk without aid 500 meters;
4.5—Fully ambulatory without aid, up and about much of day, able to work a full day, may otherwise have some limitations of full activity or require minimal assistance. Relatively severe disability. Able to walk without aid 300 meters;
5.0—Ambulatory without aid for about 200 meters. Disability impairs full daily activities;
5.5—Ambulatory for 100 meters, disability precludes full daily activities;
6.0—Intermittent or unilateral constant assistance (cane, crutch or brace) required to walk 100 meters with or without resting;
6.5—Constant bilateral support (cane, crutch or braces) required to walk 20 meters without resting;
7.0—Unable to walk beyond 5 meters even with aid, essentially restricted to wheelchair, wheels self, transfers alone; active in wheelchair about 12 hours a day;
7.5—Unable to take more than a few steps, restricted to wheelchair, may need aid to transfer; wheels self, but may require motorized chair for full day's activities;
8.0—Essentially restricted to bed, chair, or wheelchair, but may be out of bed much of day; retains self care functions, generally effective use of arms;
8.5—Essentially restricted to bed much of day, some effective use of arms, retains some self care functions;
9.0—Helpless bed patient, can communicate and eat;
9.5—Unable to communicate effectively or eat/swallow;
10.0—Death.

The functional system (FS) scale refers to the following (Kurtzke, Neurology, 1983, 33:1444-52):

CNS areas regulating body functions: Pyramidal (ability to walk), Cerebellar (Coordination), Brain Stem (Speech and Swallowing), Sensory (Touch and Pain), Bowel and Bladder; Visual; Mental; and “Other” (includes any other Neurological findings due to Multiple Sclerosis). Each Functional System (FS) is graded to the nearest possible grade, and V indicates an unknown abnormality; these are not additive scores and are only used for comparison of individual items.

### Pyramidal Function

0—Normal
1—Abnormal Signs without Disability;
2—Minimal disability;
3—Mild/Moderate ParaParesis of HemiParesis; Severe MonoParesis;
4—Marked ParaParesis or HemiParesis; Moderate QuadraParesis or MonoParesis;
5—Paraplegia, Hemiplegia, or Marked ParaParesis;
6—Quadriplegia;
V—Unknown.

### Cerebellar Function

0—Normal;
1—Abnormal Signs without disability;
2—Mild Ataxia;
3—Moderate Truncal or Limb Ataxia;
4—Severe Ataxia;
5—Unable to perform Coordinated Movements;
V—Unknown;
X—Weakness.

### Brain Stem Function

0—Normal;
1—Signs only;
2—Moderate Nystagmus or other mild disability;
3—Severe Nystagmus, Marked ExtraOcular Weakness or moderate disability of other Cranial Nerves;
4—Marked Dysarthria or other marked disability;
5—Inability to Speak or Swallow;
V—Unknown.

### Sensory Function

0—Normal;
1—Vibration or Figure—Writing decrease only, in 1 or 2 limbs;
2—Mild decrease in Touch or Pain or Position Sense, and/or moderate decrease in Vibration in 1 or 2 limb, or Vibration in 3 or 4 limbs;
3—Moderate decrease in Touch or Pain or Proprioception, and/or essentially lost Vibration in 1 or 2 limbs; or mild decrease in Touch or Pain and/or moderate decrease in all Proprioceptive tests in 3 or 4 limbs;
4—Marked decrease in Touch or Pain or loss of Proprioception, alone or combined in 1 or 2 limbs; or moderate decrease in Touch or Pain and/or severe Proprioceptive decrease in more than two limbs;
5—Loss of Sensation in 1 or 2 limbs; or moderate decrease in Touch or Pain and/or loss of Proprioception for most of the body below the head;
6—Sensation essentially lost below the head;
V—Unknown.

### Bowel and Bladder Function

0—Normal;
1—Mild Urinary Hesitancy, Urgency, or Retention;
2—Moderate Hesitancy, Urgency, or Retention of Bowel or Bladder, or rare Urinary InContinence;
3—Frequent Urinary InContinence;
4—Almost constant Cathaterization;
5—Loss of Bladder function;
6—Loss of Bowel function;
V—Unknown.

### Visual Function

0—Normal;
1—Scotoma with Visual Acuity>20/30 (corrected);
2—Worse Eye with Scotoma with maximal Acuity 20/30 to 20/59;
3—Worse Eye with large Scotoma or decrease in fields, Acuity 20/60 to 20/99;
4—Marked decrease in fields, Acuity 20/100 to 20/200; grade 3 plus maximal Acuity of better Eye<20/60;
5—Worse Eye Acuity<20/200; grade 4 plus better Eye Acuity<20/60;
V—Unknown.

### Cerebral Function

0—Normal;
1—Mood alteration;
2—Mild decrease in Mentation;
3—Moderate decrease in Mentation;
4—Marked decrease in Mentation;
5—Dementia;
V—Unknown.

### Other Function

0—Normal;
1—Other Neurological finding.

The multiple sclerosis functional composite (MSFC) is a frequently applied rating scale as well (e.g. Rudick et al., 2001). It assesses the following abilities of an MS patient: two trials of timed 25-Foot Walk; two trials of Dominant Hand by 9-HPT (9 hole peg test); two trials of Non-Dominant Hand by 9-HPT; Paced auditory serial addition test (PASAT-3″). These tests are being carried out e.g. according to a Manual prepared by Fischer et al., 2001, published by the National Multiple Sclerosis Society, or as described in Fisher J S et al., Administration and Scoring Manual for the Multiple Sclerosis Functional Composite Measure (MSFC). New York: Demos Medical Publishing, 1999.

In accordance with the present invention, TACI-Ig is used for treatment of optic neuritis. Said TACI-immunoglobulin (TACI-Ig) fusion protein comprises or consists of (a) the TACI extracellular domain or a variant or fragment thereof which binds to BLyS and/or APRIL; and (b) a immunoglobulin-constant domain.

In the frame of the present invention, the term “TACI extracellular domain” also refers to any variant thereof being at least 80% or 85%, preferably at least 90% or 95% or 99% identical to TACI the extracellular domain (SEQ ID NO: 1). The term “TACI extracellular domain” also includes variants comprising no more than 50 or 40 or 30 or 20 or 10 or 5 or 3 or 2 or 1 conservative amino acid substitutions. Any such variant is able to bind BLyS and/or APRIL and/or any BLyS-APRIL heterotrimer. Preferably, such a variant also inhibits the biological activity of BLyS and/or of APRIL and/or of any BLyS/APRIL heterotrimer. One biological activity of BLyS or APRIL is B cell proliferation, for instance.

Fragments (active fragments) and variants of the TACI extracellular domain can be used in the context of the present invention as well, as long as the fragment is able to bind BLyS and/or APRIL and/or any BLyS-APRIL heterotrimer. Preferably, such a fragment also inhibits or reduces the biological activity of BLyS and/or of APRIL and/or of any BLyS/APRIL heterotrimer.

The ability of any TACI extracellular domain, TACI-Ig fusion protein, or any variant or fragment thereof to bind BLyS and/or APRIL and/or Blys/APRIL heterotrimer can be assessed e.g. in accordance with Example 2 below. The ability to inhibit or reduce BLyS, APRIL or BLyS/APRIL heterotrimer biological activity can be assessed e.g. in accordance with Example 3 below.

It is preferred, in the context of the present invention, that any such fragment or variant of a TACI extracellular domain or a TACI-Ig fusion protein, does not have any biological activity which is significantly lower that that of atacicept, i.e. a protein having the amino acid sequence of SEQ ID NO: 3.

The term “immunoglobulin (Ig)-constant domain”, as used herein, is also called an “Fc domain” and is derived from a human or animal immunoglobulin (Ig) that is preferably an IgG. The IgG may be an IgG1, IgG2, IgG3 or IgG4. The Fc domain preferably comprises at least the CH2, CH3 domain of IgG1, preferably together with the hinge region.

In another embodiment of the invention, the Ig constant domain is a human IgG1 domain.

In one embodiment, human IgG1 constant domain has been modified for reduced complement dependent cytotoxicity (CDC) and/or antibody dependent cellular cytotoxicity (ADCC).

In ADCC, the Fc domain of an antibody binds to Fc receptors (FcγRs) on the surface of immune effector cells such as natural killers and macrophages, leading to the phagocytosis or lysis of the targeted cells. In CDC, the antibodies kill the targeted cells by triggering the complement cascade at the cell surface. The binding of IgG to the activating (FcγRI, FcγRIIa, FcγRIIIa and FcγRIIIb) and inhibitory (FcγRIIb) FcγRs or the first component of complement (C1q) depends on residues located in the hinge region and the CH2 domain. Two regions of the CH2 domain are important for FcγRs and complement C1q binding, and have unique sequences in IgG2 and IgG4. For instance, substitution of IgG2 residues at positions 233-236 into human IgG1 greatly reduced ADCC and CDC (Armour et al., 1999 and Shields et al., 2001). The following Fc mutations, according to EU index positions (Kabat et al., 1991), can e.g. be introduced into an Fc derived from IgG1:

### T250Q/M428L

### M252Y/S254T/T256E+H433K/N434F

### E233P/L234V/L235A/ΔG236+A327G/A330S/P331S

### E333A; K322A.

Further Fc mutations may e.g. be the substitutions at EU index positions selected from 330, 331 234, or 235, or combinations thereof. An amino acid substitution at EU index position 297 located in the CH2 domain may also be introduced into the Fc domain in the context of the present invention, eliminating a potential site of N-linked carbohydrate attachment. The cysteine residue at EU index position 220 may also be replaced with a serine residue, eliminating the cysteine residue that normally forms disulfide bonds with the immunoglobulin light chain constant region.

Particular Fc domains suitable for TACI-Ig fusion proteins to be used in accordance with the present invention have been prepared.

Specifically, six versions of a modified human IgG1 Fc were generated for creating Fc fusion proteins and are named Fc-488, as well as Fc4, Fc5, Fc6, Fc7, and Fc8. Fc-488 (having a DNA sequence of SEQ ID NO: 4 and an amino acid sequence of SEQ ID NO: 5) was designed for convenient cloning of a fusion protein containing the human γ1 Fc region, and it was constructed using the wild-type human immunoglobulin γ1 constant region as a template. Concern about potential deleterious effects due to an unpaired cysteine residue led to the decision to replace the cysteine that normally disulfide bonds with the immunoglobulin light chain constant region with a serine residue. An additional change was introduced at the codon encoding EU index position 218 to introduce a BgIII restriction enzyme recognition site for ease of future DNA manipulations. These changes were introduced into the PCR product encoded on the PCR primers. Due to the location of the BgIII site and in order to complete the Fc hinge region, codons for EU index positions 216 and 217 were incorporated in the fusion protein partner sequences.

Fc4, Fc5, and Fc6 contain mutations to reduce effector functions mediated by the Fc by reducing FcγRI binding and complement C1q binding. Fc4 contains the same amino acid substitutions that were introduced into Fc-488. Additional amino acid substitutions were introduced to reduce potential Fc mediated effector functions. Specifically, three amino acid substitutions were introduced to reduce FcγRI binding. These are the substitutions at EU index positions 234, 235, and 237. Substitutions at these positions have been shown to reduce binding to FcγRI (Duncan et al., 1988). These amino acid substitutions may also reduce FcγRIIa binding, as well as FcγRIII binding (Sondermann et al., 2000; Wines et al., 2000).

Several groups have described the relevance of EU index positions 330 and 331 in complement C1q binding and subsequent complement fixation (Canfield and Morrison, 1991; Tao et al., 1993). Amino acid substitutions at these positions were introduced in Fc4 to reduce complement fixation. The C_{H3} domain of Fc4 is identical to that found in the corresponding wild-type polypeptide, except for the stop codon, which was changed from TGA to TAA to eliminate a potential dam methylation site when the cloned DNA is grown in dam plus strains of E. coli.

In Fc5, the arginine residue at EU index position 218 was mutated back to a lysine, because the BgIII cloning scheme was not used in fusion proteins containing this particular Fc. The remainder of the Fc5 sequence matches the above description for Fc4.

Fc6 is identical to Fc5 except that the carboxyl terminal lysine codon has been eliminated. The C-terminal lysine of mature immunoglobulins is often removed from mature immunoglobulins post-translationally prior to secretion from B-cells, or removed during serum circulation. Consequently, the C-terminal lysine residue is typically not found on circulating antibodies. As in Fc4 and Fc5 above, the stop codon in the Fc6 sequence was changed to TAA.

Fc7 is identical to the wild-type γ1 Fc except for an amino acid substitution at EU index position 297 located in the C_{H2} domain. EU index position Asn-297 is a site of N-linked carbohydrate attachment. N-linked carbohydrate introduces a potential source of variability in a recombinantly expressed protein due to potential batch-to-batch variations in the carbohydrate structure. In an attempt to eliminate this potential variability, Asn-297 was mutated to a glutamine residue to prevent the attachment of N-linked carbohydrate at that residue position. The carbohydrate at residue 297 is also involved in Fc binding to the FcRIII (Sondermann et al., Nature 406:267 (2000)). Therefore, removal of the carbohydrate should decrease binding of recombinant Fc7 containing fusion proteins to the FcγRs in general. As above, the stop codon in the Fc7 sequence was mutated to TAA.

Fc8 is identical to the wild-type immunoglobulin γ1 region shown in SEQ ID NO:4, except that the cysteine residue at EU index position 220 was replaced with a serine residue. This mutation eliminated the cysteine residue that normally disulfide bonds with the immunoglobulin light chain constant region.

The use of any of these specific Fc domains for formation of an TACI-Ig fusion protein is within the scope of the present invention.

The immunoglobulin constant domain of TACI-Ig preferably comprises or consists of a polypeptide having an amino acid sequence of SEQ ID NO: 2, or a variant thereof being at least 80% or 85%, preferably at least 90% or 95% or 99% identical to the Ig constant domain of SEQ ID NO: 2, or a variant thereof comprising less than 50 or 40 or 30 or 20 or 10 or 5 or 3 or 2 conservative amino acid substitutions, as long as there is no impact on the overall biological activity of the TACI-Ig fusion protein, and the immunogenicity of the TACI-Ig protein is not significantly higher that that of atacicept (SEQ ID NO: 3).

In the context of the present invention, the term “identity” reflects a relationship between two or more polypeptide sequences, determined by comparing the sequences. In general, identity refers to an exact amino acid to amino acid correspondence of the two polypeptide sequences, respectively, over the length of the sequences being compared.

For sequences where there is not an exact correspondence, a “% identity” may be determined. In general, the two sequences to be compared are aligned to give a maximum correlation between the sequences. This may include inserting “gaps” in either one or both sequences, to enhance the degree of alignment. A % identity may be determined over the whole length of each of the sequences being compared (so-called global alignment), that is particularly suitable for sequences of the same or very similar length, or over shorter, defined lengths (so-called local alignment), that is more suitable for sequences of unequal length.

Methods for comparing the identity of two or more sequences are well known in the art. Thus for instance, programs available in the Wisconsin Sequence Analysis Package, version 9.1 (Devereux J et al., 1984), for example the programs BESTFIT and GAP, may be used to determine the % identity between two polynucleotides and the % identity between two polypeptide sequences. BESTFIT uses the “local homology” algorithm of Smith and Waterman (1981) and finds the best single region of similarity between two sequences. Other programs for determining identity sequences are also known in the art, for instance the BLAST family of programs (Altschul S F et al, 1990, Altschul S F et al, 1997, accessible through the home page of the NCBI at www.ncbi.nlm.nih.gov) and FASTA (Pearson W R, 1990).

Preferred amino acid substitutions in accordance with the present invention are what are known as “conservative” substitutions. Conservative amino acid substitutions of the extracellular domain of TACI or the immunoglobulin constant domain portion of the TACI-Ig fusion protein, include synonymous amino acids within a group which have sufficiently similar physicochemical properties that substitution between members of the group will preserve the biological function of the molecule (Grantham, 1974). It is clear that insertions and deletions of amino acids may also be made in the above-defined sequences without altering their function, particularly if the insertions or deletions only involve a few amino acids, e.g., under 50 or under 30, under 20, or preferably under 10 or under 5 amino acid residues, and do not remove or displace amino acids which are critical to a functional conformation, such as e.g. cysteine residues. Proteins and variants produced by such deletions and/or insertions can be used for treatment of relapsing MS as long as its biological activity is not significantly lower than the biological activity of atacicept (a protein having an amino acid sequence of SEQ ID NO: 3).

International patent applications published as WO 00/40716 and WO 02/094852 disclose sequences for the extracellular domain of TACI as well as specific fragments of the TACI extracellular domain that interact with its ligands, BLyS and APRIL.

As disclosed e.g. in WO 00/40716, the TACI extracellular domain comprises two cysteine (Cys)-rich repeats which are characteristic for members of the tumor necrosis factor (TNF) receptor superfamily, to which the TACI receptor belongs. In WO 00/40716, it has also been established that a splice variant of TACI, designated BR42×2, comprising only the second, less conserved Cys-rich repeat, was able to bind to BLyS. Therefore, in the frame of the present invention, the TACI extracellular domain fragment preferably at least comprises or consists of amino acid residues 71 to 104 of SEQ ID NO: 1, corresponding to the second Cys-rich repeat. It is further preferred that the TACI-Ig fusion protein further comprises amino acid residues 34 to 66 of SEQ ID NO: 1, corresponding to the first Cys-rich repeat.

In a further embodiment of the present invention, said TACI extracellular domain fragment, which binds to and inhibits BLyS and/or APRIL activity, comprises or consists of amino acid residues 30 to 110 of SEQ ID NO: 1.

In yet a further embodiment of the invention, the TACI-Ig fusion protein comprises or consists of a polypeptide having the sequence of SEQ ID NO: 3, or a variant thereof being at least 90% or 95% or 98% or 99% identical thereto or having less than 30 or 20 or 15 or 10 or 5 or 3 or 2 conservative amino acid substitutions, the variant binding to BlyS and/or APRIL.

In yet a further embodiment of the invention, the TACI-Ig fusion protein comprises or consists of a polypeptide having the sequence of SEQ ID NO: 8, or a variant thereof being at least 90% or 95% or 98% or 99% identical thereto or having less than 30 or 20 or 15 or 10 or 5 or 3 or 2 conservative amino acid substitutions, the variant binding to BlyS and/or APRIL.

In yet a further embodiment of the invention, the TACI-Ig fusion protein comprises or consists of a polypeptide having the sequence of SEQ ID NO: 10, or a variant thereof being at least 90% or 95% or 98% or 99% identical thereto or having less than 30 or 20 or 15 or 10 or 5 or 3 or 2 conservative amino acid substitutions, the variant binding to BlyS and/or APRIL.

In yet a further embodiment of the invention, the TACI-Ig fusion protein comprises or consists of a polypeptide having the sequence of SEQ ID NO: 12, or a variant thereof being at least 90% or 95% or 98% or 99% identical thereto or having less than 30 or 20 or 15 or 10 or 5 or 3 or 2 conservative amino acid substitutions, the variant binding to BlyS and/or APRIL.

In yet a further embodiment of the invention, the TACI-Ig fusion protein comprises or consists of a polypeptide having the sequence of SEQ ID NO: 14, or a variant thereof being at least 90% or 95% or 98% or 99% identical thereto or having less than 30 or 20 or 15 or 10 or 5 or 3 or 2 conservative amino acid substitutions, the variant binding to BlyS and/or APRIL.

The dosing of TACI-Ig fusion protein for treatment of relapsing multiple sclerosis is preferably in the range of about 10 to about 400 mg per person per week, more preferably in the range of about 20 to about 300 mg per person per week.

In an embodiment of the present invention, the TACI-Ig fusion protein is prepared or formulated for administration in amount of 25 or 75 or 150 mg per patient per week. In an embodiment, the TACI-Ig fusion protein is administered once per week in an amount of 150 mg per patient. Preferably, the TACI-Ig fusion protein is administered in an amount of 150 mg per patient per week.

In another preferred embodiment, the TACI-Ig fusion protein is administered twice per week in an amount of 50 or 150 or 300 mg per patient. Preferably, the TACI-Ig fusion protein is administered in an amount of 300 mg per patient per week.

The use of numerical values in the various ranges specified in this application, unless expressly indicated otherwise, are stated as approximations as though the minimum and maximum values within the stated ranges were both preceded by the word “about.” In this manner, slight variations above and below the stated ranges can be used to achieve substantially the same results as values within the ranges. Also, the disclosure of ranges is intended as a continuous range including every value between the minimum and maximum values recited as well as any ranges that can be formable thereby.

The TACI-Ig fusion protein may be prepared or formulated for administration every day or every other day, preferably twice a week or weekly. Preferably, the administration of TACI-Ig is a bolus administration once per week.

In another embodiment, the TACI-Ig fusion protein is prepared or formulated for administration every other week or once per month.

In one embodiment, the TACI-Ig fusion protein is prepared or formulated for administration twice a week (biweekly) during a loading period. During the loading period, the TACI-Ig fusion protein is preferably administered in an amount of 300 mg per patient per week. In a further embodiment, the TACI-Ig fusion protein is prepared or formulated for administration once per week (weekly) during a maintenance period.

During the maintenance period, the TACI-Ig fusion protein is preferably administered in an amount of 150 mg per patient per week.

In accordance with an embodiment of present invention, the loading period is preferably at least 1, 2 or 3 weeks and preferably up to one month and the maintenance period is preferably at least 1 or 2 or 3 or 5 or 6 or 7 or 8 or 9 or 10 or 12 or 13 or 14 or 15 or 16 or 17 or 18 months.

The TACI-Ig fusion protein can be formulated e.g. for intravenous, subcutaneous, or intramuscular routes.

In an embodiment of the invention, the TACI-Ig fusion protein is prepared or formulated for a subcutaneous administration.

For parenteral (e.g. intravenous, subcutaneous, intramuscular) administration, the TACI-Ig fusion protein can be formulated as a solution, suspension, emulsion or lyophilized powder in association with a pharmaceutically acceptable parenteral vehicle (e.g. water, saline, dextrose solution) and additives that maintain isotonicity (e.g. mannitol) or chemical stability (e.g. preservatives and buffers). The formulation is sterilized by commonly used techniques.

In an embodiment of the invention, the TACI-Ig fusion protein is in a formulation comprising sodium acetate buffer and trehalose, preferably in a 10 mM sodium acetate buffer at about pH5.

In a further embodiment, the invention relates to a method of treating optic neuritis, preferably as clinically isolated syndrome, and to a method of preventing development of relapsing MS or recurrent optic neuritis attacks, comprising administering to a patient a composition comprising a fusion molecule comprising:

a) the TACI extracellular domain or a fragment or variant thereof thereof which binds BlyS; and
b) a human immunoglobulin-constant domain, or a fragment or variant thereof, in amount effective to treat said relapsing multiple sclerosis.

The invention further relates to uses and methods of treating optic neuritis, and in particular of recurrent optic neuritis such as a second optic neuritis attack in the same eye, or of relapses in patients converting to CDMS, with a TACI-Ig fusion protein in combination with a corticosteroid. The corticosteroid is preferably methylprednisolone. In an embodiment, methylprednisolone is used at 1000 mg per patient per day intravenously.

Methods and uses in accordance with the present invention can be combined with other methods of treatment for relapsing multiple sclerosis, such as treatment with interferon-beta, cladribine, mitoxantrone, glatiramer acetate, natalizumab, rituximab, teriflunomide, fingolimod, laquinimod, or BG-12. The combined treatment can be simultaneous, separate or sequential.

Having now fully described this invention, it will be appreciated by those skilled in the art that the same can be performed within a wide range of equivalent parameters, concentrations and conditions without departing from the spirit and scope of the invention and without undue experimentation.

While this invention has been described in connection with specific embodiments thereof, it will be understood that it is capable of further modifications. This application is intended to cover any variations, uses or adaptations of the invention following, in general, the principles of the invention and including such departures from the present disclosure as come within known or customary practice within the art to which the invention pertains and as may be applied to the essential features hereinbefore set forth as follows in the scope of the appended claims.

All references cited herein, including journal articles or abstracts, published or unpublished U.S. or foreign patent application, issued U.S. or foreign patents or any other references, are entirely incorporated by reference herein, including all data, tables, figures and text presented in the cited references. Additionally, the entire contents of the references cited within the references cited herein are also entirely incorporated by reference.

Reference to known method steps, conventional methods steps, known methods or conventional methods is not any way an admission that any aspect, description or embodiment of the present invention is disclosed, taught or suggested in the relevant art.

The foregoing description of the specific embodiments will so fully reveal the general nature of the invention that others can, by applying knowledge within the skill of the art (including the contents of the references cited herein), readily modify and/or adapt for various application such specific embodiments, without undue experimentation, without departing from the general concept of the present invention. Therefore, such adaptations and modifications are intended to be within the meaning and range of equivalents of the disclosed embodiments, based on the teaching and guidance presented herein. It is to be understood that the phraseology or terminology herein is for the purpose of description and not of limitation, such that the terminology or phraseology of the present specification is to be interpreted by the skilled artisan in light of the teachings and guidance presented herein, in combination with the knowledge of one of ordinary skill in the art.

Having now described the invention, it will be more readily understood by reference to the following example of an exemplary clinical study outline that is provided by way of illustration, and not intended to be limiting of the present invention.

### Example 1

### A Two-Arm, Randomized, Double-Blind, Placebo-Controlled, Multicenter Phase II Study to Evaluate Safety and Tolerability and to Explore the Neuroprotective Effect of Atacicept as Assessed by Optical Coherence Tomography (OCT) in Subjects with Optic Neuritis (ON) as Clinically Isolated Syndrome (CIS) Over a 36 Week Treatment Course

### List of Abbreviations

AE Adverse Event
ALT Alanine Aminotransferase
ANCOVA Analysis of Covariance
AP Alkaline Phosphatase
APRIL A proliferation-inducing ligand
AST Aspartate Aminotransferase
BCMA B cell maturation antigen
BIW Twice weekly
BLyS B-lymphocyte stimulator
CA Competent Authorities
CDMS Clinically Definite MS
CI Confidence Interval
CIS Clinically Isolated Syndrome
CJD Creutzfeldt-Jakob disease
CNS Central Nervous System
CQA Corporate Quality Assurance
CRF Case Report Form
CRO Clinical Research Organisation
CRP C-reactive Protein
CTCAE Common Terminology Criteria for Adverse Events
CTS Clinical Trial Supplies
DMD Disease-Modifying Drug
DMPK Drug Metabolism and Pharmacokinetics
DMC Data Monitoring Committee
DQA Development Quality Assurance
DST Data Standards Team
EC Ethics Committee
ECG Electrocardiogram
ECRF Electronic Case Report Form
EDSS Expanded Disability Status Score
ESR Erythrocyte Sedimentation Rate
ETDRS Early Treatment Diabetic Retinopathy Study
EU European Union
FDA Food and Drug Administration
GCP Good Clinical Practice
Gd Gadolinium
GEE Generalized Estimating Equation
GDS Global Drug Safety
HBsAg Hepatitis B surface antigen
HIPAA Health Insurance Portability and Accountability Act
HIV Human immunodeficiency virus
IB Investigator Brochure
ICH International Conference on Harmonisation
IEC Independent Ethics Committee
IMP Investigational Medicinal Product
IRB Independent Review Board
ITT Intention to Treat
IUD Intra Uterine Device
IVIg Intravenous Immunoglobulin
IVRS Interactive Voice Response System
KFS Kurtzke Functional Systems
LD Loading Dose
LPLV Last Patient Last Visit
mcg Microgram
MD Maintenance dose
ml Millilitre
MRI Magnetic Resonance Imaging
MRI-AC Magnetic Resonance Imaging Analysis Centre
MS Multiple Sclerosis
NYHA New York Health Association
OCT Optical Coherence Tomography
ON Optic Neuritis
PCFR Parent-Child/Foetus Report
PD Pharmacodynamics
PGx Pharmacogenetics/Pharmacogenomics
PK Pharmacokinetics
PP Per Protocol
QW Once Weekly
R&D Research and Development
RA Rheumatoid Arthritis
RD Relative Difference
RGC Retinal Ganglion Cell
RMS Relapsing Multiple Sclerosis
RNFL Retinal Nerve Fiber Layer
RoW Rest of the World
SAE Serious Adverse Event
SAP Statistical Analysis Plan
sc Subcutaneous(ly)
SD1 Study Day 1
SEC Safety and Ethics Committee
SLE Systemic Lupus Erythematosus
SOP Standard Operating Procedure
SRB Safety Review Board
SUSAR Suspected Unexpected Serious Adverse Reaction
TACI Transmembrane activator and calcium modulatorfor and cyclophilin-ligand (CAML)-interactor
TD Treatment Dose
TIW Three times a week
TNF Tumor Necrosis Factor
ULN Upper Limit of Normal
WBC White Blood Cell Count

### Study Synopsis

### Objectives:

### Primary Objective:

Evaluate the efficacy of atacicept to preserve Retinal Nerve Fiber Layer (RNFL) thickness in ON as assessed by Optical Coherence Tomography (OCT).

### Secondary Objectives:

Evaluate safety and tolerability of atacicept in subjects with ON including the incidence and severity of infections and the conversion of subjects with ON to RMS as per McDonald criteria or to Clinically Definite MS (CDMS).

Explore the effect of atacicept on visual outcomes such as low contrast letter acuity and contrast sensitivity in subjects with ON.

### Tertiary and Exploratory Objectives:

Obtain further information on the involvement of B cell immunity in the pathology of ON by correlating the pharmacodynamic (PD) profile of atacicept in ON subjects with RNFL preservation and visual outcomes.

Explore the effect of atacicept on visual function such as colour vision, visual field and high contrast sensitivity.

Perform pharmacogenetic/pharmacogenomic studies in a subset of subjects to identify possible associations between gene polymorphisms or gene expression profiles and drug response, respectively.

Evaluate the pharmacokinetics of atacicept for 36 weeks, given at 150 mg SC weekly (QW), preceded by a loading phase of 150 mg SC twice a week (BIW) during the first 4 weeks of the 36-week treatment course.

### Endpoints:

### Primary Endpoint:

The primary endpoint is the change of RNFL thickness in the affected eye of ON patients from Baseline to week 36, assessed by OCT.

### Secondary Endpoints:

### Efficacy Endpoints

Difference in RNFL thickness between the affected eye and fellow eye in ON patients at weeks 12, 24 and 36
Change of RNFL thickness in the affected eye of ON patients from Baseline to weeks 12 and 24
Change in macular thickness at 3 mm around fovea in the affected eye of ON patients from Baseline to weeks 12, 24 and 36
Change in macular thickness at 6 mm around fovea in the affected eye of ON patients from Baseline to weeks 12, 24 and 36
Change in macular volume in the affected eye of ON patients from Baseline to weeks 12, 24 and 36
Low contrast letter acuity (Sloan charts) at weeks 12, 24 and 36
Contrast sensitivity (Pelli-Robson charts) at weeks 12, 24 and 36

### Safety Endpoints

Nature, severity, and incidence of adverse events including infections
Incidence and severity of laboratory abnormalities
Injection site reactions
Changes in vital signs, ECGs
Proportion of subjects who develop antibodies to atacicept during the course of the study
Proportion of subjects converting to RMS as per McDonald criteria or CDMS (second clinical attack) during the 36 week treatment period
EDSS change (relative to baseline) at week 36.

### Tertiary/Exploratory Endpoints:

High contrast letter acuity (Early Treatment Diabetic Retinopathy Study (ETDRS) chart) at weeks 12, 24 and 36
Automated visual field (Humphrey automated perimetry) at weeks 12, 24 and 36
Color vision (Farnsworth Munsell D15 test) at weeks 12, 24 and 36
Pharmacokinetic (PK) measures: free atacicept, composite atacicept (free atacicept+atacicept BLys complex), total atacicept (free atacicept+atacicept-BLyS complex+atacicept-APRIL complex), atacicept-BLyS complex
Pharmacodynamic (PD) measures: Free APRIL and free BLyS (contingent on availability of appropriate assays for post-dose samples), ESR, CRP, total immunoglobulin isotypes, lymphocyte subpopulations
In a subset of subjects pharmacogenomic/pharmacogenetic (PGx) studies will be performed to identify possible association between gene polymorphism or gene expression profile and drug response, respectively

This is a two-arm, randomized, double-blind, placebo-controlled multicenter Phase II study to evaluate safety and tolerability and to explore the neuroprotective effect of atacicept as assessed by OCT vs. matching placebo in ON subjects over a 36 week treatment course.

Subjects will receive atacicept or placebo at a 1:1 randomization ratio. Atacicept will be given at 150 mg SC weekly (QW), preceded by a loading dose of 150 mg SC twice a week (BIW) during the first 4 weeks of the 36-week treatment course. The control group will receive matching placebo. During this study, there will be one screening visit (within 28 days prior to Study Day 1 (SD1) visit), a SD1 visit at which time subjects will be randomized and study treatments initiated, and subsequent visits at weeks 1, 4, 8, 12, 16, 20 24, 30 and 36. Regular telephone contacts will be implemented between scheduled visits. The use of corticosteroids will be optional for the treatment of the initial ON event. Subsequent use of corticosteroid will be limited to the treatment of relapses in patients converting to CDMS as defined in Appendix B or in patients developing a second ON attack in the same eye.

The subjects will be followed-up for 12 weeks after the last dose. OCT, visual function and safety assessments will be performed at the Follow-up visit at week 48.

For all randomized subjects, there will be a rescue option of treatment with Rebif® (44 mcg three times a week (tiw) for the course of the study, if subjects convert to CDMS and if the investigator considers the treatment with disease modifying drugs indicated. Any subject accepting rescue medication will be withdrawn from IMP, but will remain in the study, performing all scheduled assessments according to the visit schedule.

### Trial Population:

Patients that are eligible for this study will have to be diagnosed with symptomatic unilateral ON as a first clinical event (clinically isolated syndrome, CIS). The inclusion criteria aim to ensure the enrolment of CIS subjects presenting with ON and avoid prior therapies that could confound the evaluation of safety and efficacy during the trial. Furthermore, past infections or comorbidities that could recur during the trial and confound the safety assessments are excluded.

ON as a first clinical manifestation of a demyelinating disease may allow for a for more robust assessment of the treatment effect due to more prominent RNFL loss in this condition than in MS patients presenting with another type of clinical attack (RNFL thickness is reduced in MS even without symptomatic visual involvement), and therefore ensures stable baseline. Selecting patients with monofocal ON avoids the risk of severe bilateral visual impairment in the trial population and avoids the inclusion of patients with another condition like neuromyelitis optica, that has been distinguished from MS also by the presence of ON that is usually bilateral, simultaneous and often severe (Cross, 2007). In line with that, it has been indicated that patients presenting with bilateral ON have less risk of progression to MS.

This study will be conducted in approximately 30 sites located worldwide.

### Eligibility Criteria:

### Inclusion Criteria:

1. Diagnosis of unilateral symptomatic optic neuritis as first clinical manifestation within 28 days between onset of symptoms and SD1;
2. Male or female between 18-60 years old, inclusive, at the time that informed consent is obtained;
3. Written informed consent, given before any study-related procedure. Subjects must have read and understood the Informed Consent Form, must fully understand the requirements of the study and must be willing to comply with all study visits and assessments.
4. Women of childbearing potential must not be breast-feeding and have a negative serum pregnancy test at initial screening and a urine pregnancy test at Study Day 1 before dosing. For the purpose of this study, women of childbearing potential are defined as all female patients after puberty unless they are post-menopausal for at least 2 years or surgically sterile.
5. Female subjects of childbearing potential must be willing to avoid pregnancy by using adequate method of contraception for 4 weeks prior to Study Day 1, during the trial and 12 weeks after the last dose of study medication. This requirement does not apply to surgically sterile subjects or to subjects who are post-menopausal for at least 2 years. Adequate contraception is defined as follows: two barrier methods, or one barrier method with a spermicide, or an intrauterine device or use of a female hormonal contraceptive.
6. Be willing and able to comply with study procedures for the duration of the study;
7. Voluntarily provide written informed consent (obtained before any trial related procedure), including, for USA, subject authorization under Health Insurance Portability and Accountability Act (HIPAA), prior to any study-related procedure that is not part of normal medical care, and with the understanding that the subject may withdraw consent at any time without prejudice to their future medical care.

### Exclusion Criteria:

1. History of ON prior to current ON attack;
2. Bilateral optic neuritis;
3. Diagnosis of MS;
4. Diagnosis of Devic's disease;
5. Co-morbid ocular condition not related to optic neuritis (ascertained by detailed history and examination, including glaucoma, hypoplasia of the optic nerve, macular hole, vitreomacular traction, diabetes, or other diseases of the optic nerve);
6. Non-evaluable OCT at screening visit due to oedema in the affected eye defined as follows:
   RNFL thickness more than 10 μm above normal in 2 or more sectors, or RNFL thickness greater than 200 μm in any of the 12 sectors;
7. Refractive error greater than ±6 diopters;
8. Any condition, including laboratory findings and findings in the medical history or in the pre-study assessments (such as, but not limited to, significant nervous system, renal, hepatic, endocrine or gastrointestinal disorders), which in the Investigator's opinion constitutes a risk or a contraindication for the subject's participation in the study or that could interfere with the study objectives, conduct or evaluation.
9. Prior treatment with B cell modulating therapies, such as rituximab or belimumab;
10. Prior exposure to immunomodulatory therapy, such as interferon beta or glatiramer acetate;
11. Prior exposure to immunosuppressive or cytotoxic agents including but not restricted to cladribine, mitoxantrone, alemtuzumab, cyclophosphamide, azathioprine, methotrexate, or natalizumab;
12. Prior myelosuppressive/cytotoxic therapy, such as lymphoid irradiation, or bone marrow transplantation;
13. Prior use of cytokine or anti-cytokine therapy, intravenous immunoglobulin (IVIg) or plasmapheresis;
14. Treatment with oral or systemic corticosteroids or adrenocorticotropic hormone within 60 days prior to SD1; except the optional corticosteroid course to treat the initial ON event;
15. Require chronic or monthly pulse corticosteroids during the study;
16. Receive immunisations with live vaccines or Ig treatments within one month prior SD 1 or need for such treatment during the study;
17. Participation in any interventional clinical trial within 2 months before SD 1 (or within 5 half-lives of the investigated compound before SD 1, whichever is longer), prior to SD1
18. Have moderate to severe renal impairment (creatinine clearance<50 ml/min; according to Cockcroft-Gault equation);
19. Allergy or hypersensitivity to gadolinium;
20. Known hypersensitivity to atacicept or to any of the components of the formulated atacicept.
21. Diagnosis or family history of Creutzfeldt-Jakob disease (CJD)
22. History or presence of uncontrolled or New York Health Association (NYHA) class 3 or 4 congestive heart failure;
23. History of cancer, except adequately treated basal cell carcinoma of the skin, cervical dysplasia or carcinoma in situ of the skin or the cervix;
24. Aspartate aminotransferase (AST), alanine aminotransferase (ALT) or alkaline phosphatase (AP) level>2.5×ULN. Total bilirubin>1.5×ULN at screening;
25. Clinically significant abnormality in any haematological test (e.g. haemoglobin<100 g/L (6.21 mmol/L), WBC<3*10⁹/L, lymphocyte count<0.8*10⁹/L, platelets <140*10⁹/L) at screening;
26. Clinically significant abnormality on chest X-ray performed within 3 months before SD1 or on ECG performed at screening;
27. Known active clinically significant acute or chronic infection, or any major episode of infection requiring hospitalisation or treatment with parenteral anti-infectives within 4 weeks of SD1 assessments;
28. Positive HIV, hepatitis C or hepatitis B (HBsAg) serology (test performed at screening);
29. Presence of active or latent tuberculosis within the past year prior to screening. Subjects should be evaluated and screened for active or latent tuberculosis according to national and/or local recommendations.
30. Serum IgG below 6 g/L at screening.

### Investigational Medicinal Product:

Atacicept drug product will be supplied as a clear to slightly opalescent, slightly yellow to yellow sterilised solution for injection in pre-filled syringes, each containing 150 mg of atacicept in a volume of 1 mL.

Placebo will be supplied as a transparent, sterile solution for injection in pre-filled syringes matching the atacicept pre-filled syringes, each containing 1 mL.

Pre-filled syringes of trial medication will be covered by non-transparent labels to prevent subjects and trial personnel from noticing any differences in the colours of the solutions.

### Data Analysis and Statistics:

### Determination of Sample Size

A total of 82 patients (41 randomized patients per arm) will provide at least 80% power to detect a difference in the primary endpoint, assuming RNFL losses at 36 weeks of 20 μm and 10 μm in the placebo and the atacicept treatment arm respectively, corresponding to a relative difference (RD) of 50%. This calculation was done assuming a two-sided Type 1 error rate of 5% and standard deviations (SD) of 20 μm in the placebo arm and 4 μm in the treatment arm. This calculation assumes a 15% non-evaluable rate. Calculations are based on a two-sample Satterthwaite t-test for unequal variances (NQuery 5.0)

### Randomization

Subjects will be randomized in a 1:1 ratio to receive either atacicept or placebo in a double-blind fashion. Subjects may be randomized only after eligibility has been confirmed. Randomization will be stratified by gender and by MRI lesions (absence or presence of MRI lesions at screening). A random permuted block design will be used to obtain balance of treatments in a 1:1 ratio within the stratification factors. Allocation to treatment group will be determined using centralized randomization through an Interactive Voice Response System (IVRS).

### Analysis Populations

The intent-to-treat (ITT) population will consist of all randomized subjects. Subjects will be analyzed according to their randomized treatment. The per protocol population consists of all randomized subjects who complete 36-weeks of treatment and are considered not to have major protocol violations. For the analysis of the primary endpoint, the per protocol population must have a valid Week 36 OCT assessment, as well as an available baseline OCT. The PP population is the primary analysis set for the primary endpoint. All efficacy endpoints will be analyzed for both the ITT and the PP population. Any differences in the conclusions between the PP and ITT analyzes will be explored and discussed. The safety population will consist of all randomized subjects with follow-up safety data who received at least one dose of the study treatment.

### Statistical Methodology

The primary endpoint of preservation of RNFL thickness at week 36 will be compared between atacicept 150 mg and placebo using a two-sided t-test for unequal variances. In the presence of extreme values or non-normality (assessed visually), the comparison between treatment groups will be done using the Wilcoxon rank-sum test as the primary method. An ANCOVA analysis including the two stratification factors (gender and screening MRI lesions (absence or presence)) will be conducted to assess if the treatment effect is influenced by these two factors. In addition, the ANCOVA with effects for region, baseline RNFL, smoking history and use of corticosteroids in the screening phase will be repeated to assess if the treatment effect is influenced by these covariate factors. Secondary and tertiary endpoints related to changes in optic nerve pathology and visual function, measured at 12, 24, and 36 weeks will be analyzed using the same approach as the primary endpoint. Descriptive statistics with 95% confidence intervals will be provided by treatment arm to assess changes in the endpoints over time. These analyzes will also serve to explore the timing of the effect of atacicept on the primary endpoint of RNFL loss.

### Investigational Medicinal Drugs

Pre-filled syringes of atacicept and placebo will be supplied by the Sponsor. Medications will be provided in treatment kits as described in Section 7.3.

### Atacicept

Atacicept drug product will be supplied as a clear to slightly opalescent, slightly yellow to yellow sterilised solution for injection in pre-filled syringes each containing 1 mL.

The formulation to be used in this trial contains atacicept at strength of 150 mg/mL, with trehalose and 10 mmol sodium acetate buffer as excipients (pH 5).

### Placebo

Placebo will be supplied as a transparent, sterile solution for injection in pre-filled syringes matching the atacicept pre-filled syringes, each containing 1 mL.

The placebo formulation to be used in this trial contains trehalose and 10 mmol sodium acetate buffer (pH 5).

### Dosage and Administration

### Eligible Subjects Will be Randomized to Receive Atacicept or Matching Placebo, Given by Subcutaneous Injection.

Treatment will consist of a loading period during the first 4 weeks, during which the assigned dose will be administered twice weekly (BIW; on Study Days 1, 4, 8, 11, 15, 18, 22 and 25) followed by a maintenance period over the next 32 weeks, during which the assigned dose will be administered once weekly (QW), beginning on week 5.

Atacicept group: atacicept 150 mg SC twice weekly (BIW) for 4 weeks, followed by 150 mg SC once weekly (QW) for 32 weeks;

Placebo group: Placebo SC twice weekly (BIW) for 4 weeks, followed by placebo SC once weekly (QW) for 32 weeks.

Atacicept and placebo will be injected SC into the anterior abdominal wall, using the provided pre-filled syringes. The volume of solution to be injected on each occasion will be 1.0 mL. Injections sites should be rotated.

### Rescue Medication

As a rescue therapy, Rebif® 44 mcg pre-filled syringes will be supplied by the Sponsor. The dosage of Rebif®, following initial dose titration, is 44 mcg administered three times a week (tiw) by sc injection. Rebif® should be stored refrigerated between 2-8° C. (36-46° F.) in a locked dispensary. The medication must not be frozen.

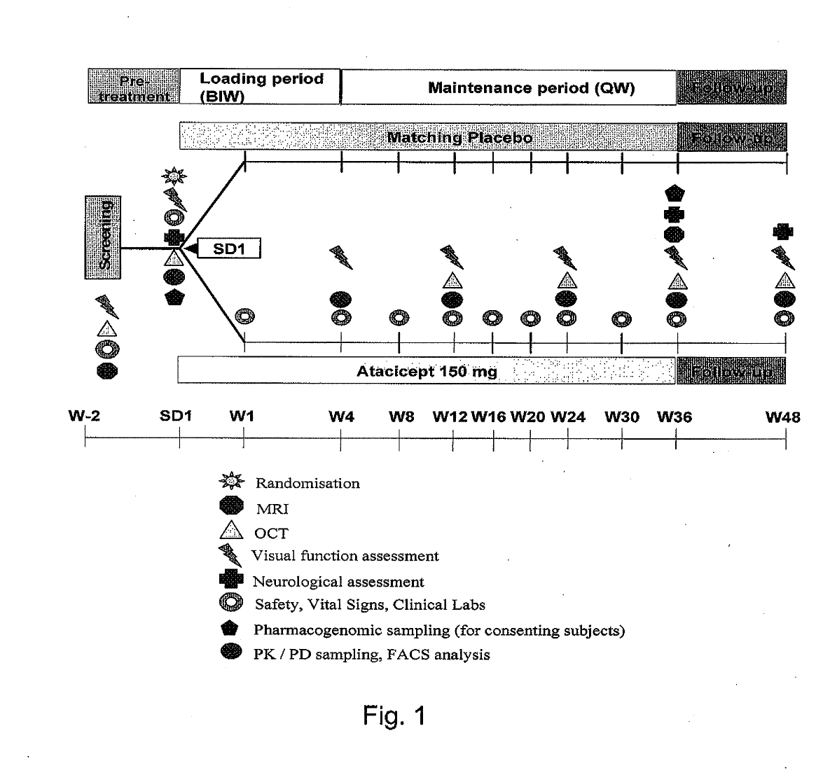

Potential side effects at the onset of treatment may be minimized by a progressive increase in the dose for the first four weeks as outlined in FIG. 1. Each dose should be recorded in the subject diary with the volume of the dose and the date and time of administration.

The Rebiject II™ autoinjector is an optional device intended for automating subcutaneous injection of Rebif® in pre-filled glass syringes, which will be provided upon request.

All subjects should be instructed by the investigative site personnel on proper medication handling, self-injection procedures, drug titration and administration, the use of the

For complete information on Rebif® administration, the local approved labeling including the patient information leaflet can be consulted.

### APPENDICES

### Methodology

### APPENDIX A

### Revised McDONALD Criteria

The revised McDonald criteria (2005) define a dissemination of the multiple sclerosis lesions in space and time as follows:

### Dissemination in Space:

Subjects should have three of the following lesions:
at least one gadolinium-enhancing lesion or nine T2-hyperintense lesions if there is no Gd-enhancing lesion.
at least one infratentorial lesion.
at least one juxtacortical lesion.
at least one periventricular lesion

NOTE: A spinal cord lesion can be considered equivalent to a brain infratentorial lesion. An enhancing spinal cord lesion is considered to be equivalent to an enhancing brain lesion, and individual spinal cord lesions can contribute together with individual brain lesions to reach the required number of T2 lesions.

### Dissemination in Time (see FIG. 2):

There are two ways to demonstrate dissemination in time using imaging:
Detection of Gd-enhancement at least 3 months after the onset of the initial clinical event, if not at the site corresponding to the initial event; and
Detection of a new T2 lesion if it appears at any time compared with the reference scan performed at least 30 days after the onset of the initial clinical event.

### APPENDIX B

### Criteria for MS Clinical Attack/Relapse

All the following criteria (a, b, c) have to be met:
Neurological abnormality, either newly appearing or re-appearing, with abnormality specified by both (i) Neurological abnormality separated by at least 30 days from onset of a preceding clinical event, and (ii) Neurological abnormality lasting for at least 24 hours.
Absence of fever or known infection (fever with temperature (measured axillary, orally or intrauriculary)>37.5° C./99.5° F.).
Objective neurological impairment, correlating with the subject's reported symptoms, defined as either i) Increase in at least one of the functional systems of the EDSS, or ii) Increase of the total EDSS score.

The occurrence of paresthesia, fatigue, mental symptoms, and/or vegetative symptoms without any additional symptom will not be classified as an MS clinical attack.

### Example 2

### Binding Assays for Testing the Binding of TACI-Ig Fusion Proteins, Variants and Fragments thereof to BLyS or April

Two approaches can be used to examine the binding characteristics of TACI-Ig fusion proteins and variants and fragments thereof (in the following: TACI-Fc constructs) with BLyS.

One approach measures the ability of the TACI-Fc constructs to compete with TACI-coated plates for binding of ¹²¹I-labeled BLyS. In the second approach, increasing concentrations of ¹²⁵I-labeled BLyS are incubated with each of the TACI-Fc constructs, and the radioactivity associated with precipitated BLyS-TACI-Fc complexes is determined.

### A. Competitive Binding Assay:

BLyS is radio-iodinated with Iodobeads (Pierce), following standard methods. Briefly, 50 μg of BLyS is iodinated with 4 mCi of 1251 using a single Iodobead. The reaction is quenched with a 0.25% solution of bovine serum albumin, and the free ¹²⁵I is removed by gel filtration using a PD-10 column (Pierce). The specific radioactivity of ¹²⁵I-BLyS preparations is determined by trichloroacetic acid precipitation before and after the desalting step.

An N-terminal fragment of the TACI receptor, designated as “TACI-N,” is added to 96-well plates (100 u. I at 0.1 ug/ml), and incubated overnight at 4° C. The plates are washed, blocked with Superblock (Pierce), and washed again. The TACI-Fc constructs, at various concentrations ranging from 0 to 11.5 ng/ml, are mixed with a fixed concentration of 1251-BLyS (20 ng/ml), and incubated for 2 hours at 37° C. on the plate coated with TACI-N. Controls contain either TACI-N in solution, or lacked TACI-Fc. After incubation, the plates are washed and counted. Each determination is performed in triplicate.

The results show whether a given TACI-Fc construct inhibits ¹²⁵I-BLyS binding completely at concentrations of about 100 ng/ml or greater and can be compared to a known TACI-Fc construct such as a construct comprising the full extracellular domain of TACI (i.e. a construct comprising SEQ ID NO: 1).

A Fc fragment alone can be tested as a further control, it does not inhibit binding.

IC50 values can be calculated for each construct in three experiments and then average values indicated.

### B. Solution Binding Assay:

At a concentration of 0.05 nM, each TACI-Fc construct is incubated with 0.4 pM to 1.5 nM ¹²⁵I-BLyS for 30 minutes at room temperature in a total volume of 0.25 ml/tube. A Pansorbin (Staph A) suspension was added to each tube, and after 15 minutes, the samples were centrifuged, washed twice, and the pellets counted.

Nonspecific binding is determined by the addition of 130 nM unlabeled BLyS to the ¹²⁵I-BLyS/TACI-Fc mix. Specific binding is calculated by subtracting the cpm bound in the presence of unlabeled BLyS from the total cpm bound at each concentration of 1251-BLyS. Each determination is performed in triplicate. Binding constants are calculated using Graph Pad Prism software (Macintosh v. 3.0).

The assays described under (A) and (B) above can be used for measurement of binding of TACI-Ig, a variant or fragment thereof to APRIL by replacing BLyS by APRIL.

### Example 3

### Human B Cell Proliferation Bioassay for Testing the Inhibition of BLyS or BLyS/April Heterotrimer Activity by TACI-Ig Fusion Proteins, Variants and Fragments Thereof

This assay is e.g. described in Roschke et al., 2002.

### Human and Murine B Cell Proliferation

Human tonsillar B cells are isolated by Ficoll centrifugation followed by negative selection using MACS magnetic beads (Miltenyi Biotec, Auburn, Calif.). Spleen cells are isolated from 6- to 10-wk-old female BALB/c mice by Ficoll centrifugation. B cell proliferation is assessed in the presence of Staphylococcus aureus cells (1/100,000 final dilution; Pansorbin; Calbiochem, La Jolla, Calif.) and protein concentrations ranging from 90 ng/ml to 0.01 pg/ml. Cells are resuspended at 1×10⁵/well in a final volume of RPMI 10% FBS containing 1×10⁻⁵ M 2-ME, and incubated in the presence of the BLyS, APRIL or BLyS/APRIL heterotrimer to be tested for 72 h. The cells are then pulsed with 0.5 μCi/well of [H³]thymidine for another 20 h. Incorporation of thymidine is used as a measure of cellular proliferation.

In order to test inhibition of BLyS, APRIL or BLyS/APRIL heterotrimer by a TACI-Ig fusion protein, variant or fragment thereof, cells are incubated in the presence of 3 ng/ml of either BLyS, APRIL or APRIL/BLyS heterotrimer, and neutralizing activity is tested at concentrations ranging from 10 pg/ml to 100 pg/ml (six 10-fold dilutions).

### Example 4

### Production of BLyS Antagonist

Four amino terminal truncated versions of TACI-Fc were generated. All four had a modified human tissue plasminogen activator signal sequence as disclosed in WO 02/094852 (SEQ ID NO: 25) fused to amino acid residue number 30 of SEQ ID NO: 6. However, the four proteins differed in the location of point in which the Fc5 was fused to the TACI amino acid sequence of SEQ ID NO: 6. Table 1 outlines the structures of the four fusion proteins.

TABLE 1
TACI Fc Fusion Proteins

| Designation of TACI-Fc | TACI amino acid residues |
|---|---|
| TACI(d1-29)-Fc5 | 30 to 154 of SEQ ID NO: 6 |
| TACI(d1-29, d107-154)-Fc5 | 30 to 106 of SEQ ID NO: 6 |
| TACI(d1-29, d111-154)-Fc5 | 30 to 110 of SEQ ID NO: 6 |
| TACI(d1-29, d120-154)-Fc5 | 30 to 119 of SEQ ID NO: 6 |

Protein encoding expression cassettes were generated by overlap PCR using standard techniques (see, for example, Horton et al., 1989). A nucleic acid molecule encoding TACI and a nucleic acid molecule encoding Fc5 were used as PCR templates. Oligonucleotide primers are identified in Tables 2 and 3.

TABLE 2
Oligonucleotide Primers Used to Produce TACI Fusion Proteins

| | Oligonucleotide Designations | | | |
|---|---|---|---|---|
| Designation of TACI-Fc | 5′ TACI | 3′ TACI | 5′ Fc5 | 3′ Fc5 |
| TACI(d1-29)-Fc5 | ZC24,903 | ZC24,955 | ZC24,952 | ZC24,946 |
| TACI(d1-29, d107-154)- | ZC24,903 | ZC24,951 | ZC24,949 | ZC24,946 |
| Fc5 | | | | |
| TACI(d1-29, d111-154)- | ZC24,903 | ZC28,978 | ZC28,979 | ZC24,946 |
| Fc5 | | | | |
| TACI(d1-29, d120-154)- | ZC24,903 | ZC28,981 | ZC28,980 | ZC24,946 |
| Fc5 | | | | |

TABLE 3
Oligonucleotide Sequences

| | | SEQ ID |
|---|---|---|
| Primer | Nucleotide Sequence | NO. |
| ZC24,903 | 5′ TATTAGGCCGGCCACCATGGATGCAATGA 3′ | 15 |
| ZC24,955 | 5′ TGAAGATTTGGGCTCCTTGAGACCTGGGA 3′ | 16 |
| ZC24,952 | 5′ TCCCAGGTCTCAAGGAGCCCAAATCTTCA 3′ | 17 |
| ZC24,946 | 5′ TAATTGGCGCGCCTCTAGATTATTTACCCGGAG | 18 |
| | ACA 3′ | |
| ZC24,951 | 5′ TGAAGATTTGGGCTCGTTCTCACAGAAGTA 3′ | 19 |
| ZC24,949 | 5′ ATACTTCTGTGAGAACGAGCCCAAATCTTC | 20 |
| | A 3′ | |
| ZC28,978 | 5′ TTTGGGCTCGCTCCTGAGCTTGTTCTCACA 3′ | 21 |
| ZC28,979 | 5′ CTCAGGAGCGAGCCCAAATCTTCAGACA 3′ | 22 |
| ZC28,981 | 5′ TTTGGGCTCCCTGAGCTCTGGTGGAA 3′ | 23 |
| ZC28,980 | 5′ GAGCTCAGGGAGCCCAAATCTTCAGACA 3′ | 24 |

The first round of PCR amplifications consisted of two reactions for each of the four amino terminal truncated versions. The two reactions were performed separately using the 5′ and 3′ TACI oligonucleotides in one reaction, and the 5′ and 3′ Fc5 oligonucleotides in another reaction for each version. The conditions of the first round PCR amplification were as follows. To a 25 μl final volume was added approximately 200 ng template DNA, 2.5 μl 10×Pfu reaction Buffer (Stratagene), 2 μl of 2.5 mM dNTPs, 0.5 μl of 20 μM each 5′ oligonucleotide and 3′ oligonucleotide, and 0.5 μl Pfu polymerase (2.5 units, Stratagene). The amplification thermal profile consisted of 94° C. for 3 minutes, 35 cycles at 94° C. for 15 seconds, 50° C. for 15 seconds, 72° C. for 2 minutes, followed by a 2 minute extension at 72° C. The reaction products were fractionated by agarose gel electrophoresis, and the bands corresponding to the predicted sizes were excised from the gel and recovered using a QIAGEN QIAQUICK Gel Extraction Kit (Qiagen), according to the manufacturer's instructions.

The second round of PCR amplification, or overlap PCR amplification reaction, was performed using the gel purified fragments from the first round PCR as DNA template. The conditions of the second round PCR amplification were as follows. To a 25 μl final volume was added approximately 10 ng template DNA each of the TACI fragment and the Fc5 fragment, 2.5 μl 10×Pfu reaction Buffer (Stratagene), 2 μl of 2.5 mM dNTPs, 0.5 μl of 20 μM each ZC24,903 (SEQ ID NO: 15) and ZC24,946 (SEQ ID NO: 18) and 0.5 μl Pfu polymerase (2.5 units, Stratagene). The amplification thermal profile consisted of 94° C. for 1 minute, 35 cycles at 94° C. for 15 seconds, 55° C. for 15 seconds, 72° C. for 2 minutes, followed by a 2 minute extension at 72° C. The reaction products were fractionated by agarose gel electrophoresis, and the bands corresponding to the predicted sizes were excised from the gel and recovered using a QIAGEN QIAQUICK Gel Extraction Kit (Qiagen), according to the manufacturer's instructions.

Each of the four versions of the amino terminal truncated TACI-Fc PCR products were separately cloned using Invitrogen's ZEROBLUNT TOPO PCR Cloning Kit following the manufacturer's recommended protocol. Table 4 identifies the nucleotide and amino acid sequences of these TACI-Fc constructs.

TABLE 4
Sequences of TACI-Fc Variants

SEQ ID Nos.

| Designation of TACI-Fc | Nucleotide | Amino Acid |
|---|---|---|
| TACI(d1-29)-Fc5 | 7 | 8 |
| TACI(d1-29, d107-154)-Fc5 | 9 | 10 |
| TACI(d1-29, d111-154)-Fc5 | 11 | 12 |
| TACI(d1-29, d120-154)-Fc5 | 13 | 14 |

After the nucleotide sequences were verified, plasmids comprising each of the four versions of the amino terminal truncated TACI-Fc fusions were digested with FseI and Ascl to release the amino acid encoding segments. The FseI-AscI fragments were ligated into a mammalian expression vector containing a CMV promoter and an SV40 poly A segment. Expression vectors were introduced into Chinese hamster ovary cells as described below.

### Example 5

### Production of TACI-FC Proteins by Chinese Hamster Ovary Cells

The TACI-Fc expression constructs were used to transfect, via electroporation, suspension-adapted Chinese hamster ovary (CHO) DG44 cells grown in animal protein-free medium (Urlaub et al., 1986). CHO DG44 cells lack a functional dihydrofolate reductase gene due to deletions at both dihydrofolate reductase chromosomal locations.

Growth of the cells in the presence of increased concentrations of methotrexate results in the amplification of the dihydrofolate reductase gene, and the linked recombinant protein-encoded gene on the expression construct.

CHO DG44 cells were passaged in PFCHO media (JRH Biosciences, Lenexa, Kans.), 4 mM L-Glutamine (JRH Biosciences), and 1× hypothanxine-thymidine supplement (Life Technologies), and the cells were incubated at 37° C. and 5% CO₂ in Corning shake flasks at 120 RPM on a rotating shaker platform. The cells were transfected separately with linearized expression plasmids. To ensure sterility, a single ethanol precipitation step was performed on ice for 25 minutes by combining 200 pg of plasmid DNA in an Eppendorf tube with 20 μl of sheared salmon sperm carrier DNA (5′→3′ Inc. Boulder, Colo., 10 mg/ml), 22 μl of 3M NaOAc (pH 5.2), and 484 μl of 100% ethanol (Gold Shield Chemical Co., Hayward, Calif.). After incubation, the tube was centrifuged at 14,000 RPM in a microfuge placed in a 4° C. cold room, the supernatant removed and the pellet washed twice with 0.5 ml of 70% ethanol and allowed to air dry.

The CHO DG44 cells were prepared while the DNA pellet was drying by centrifuging 10⁶ total cells (16.5 ml) in a 25 ml conical centrifuge tube at 900 RPM for 5 minutes. The CHO DG44 cells were resuspended into a total volume of 300 μl of PFCHO growth media, and placed in a Gene-Pulser Cuvette with a 0.4 cm electrode gap (Bio-Rad). The DNA, after approximately 50 minutes of drying time, was resuspended into 500 μl of PFCHO growth media and added to the cells in the cuvette so that the total volume did not exceed 800 μl and was allowed to sit at room temperature for 5 minutes to decrease bubble formation. The cuvette was placed in a BioRad Gene Pulser II unit set at 0.296 kV (kilovolts) and 0.950 HC (high capacitance) and electroporated immediately.

The cells were incubated 5 minutes at room temperature before placement in 20 ml total volume of PFCHO media in a CoStar T-75 flask. The flask was placed at 37° C. and 5% CO₂ for 48 hours when the cells were then counted by hemocytometer utilizing trypan blue exclusion and put into PFCHO selection media without hypothanxine-thymidine supplement and containing 200 mM methotrexate (Cal Biochem).

Upon recovery of the methotrexate selection process, the conditioned media containing the secreted TACI-Fc proteins were examined by Western Blot analysis.

### REFERENCES

Altschul S F et al, J Mol Biol, 215, 403-410, 1990.
Altschul S F et al, Nucleic Acids Res., 25:389-3402, 1997.
Antel J, Bar-Or A. Roles of immunoglobulins and B cells in multiple sclerosis: from pathogenesis to treatment. J Neuroimmunol 2006; 180:3-8.
Armour K L. et al., 1999. Recombinant human IgG molecules lacking Fcgamma receptor I binding and monocyte triggering activities. Eur J. Immunol. 29(8):2613-24.
Balcer L J, Baier M L, Cohen J A, Kooijmans M F, Sandrock A W, Nano-Schiavi M L et al. Contrast letter acuity as a visual component for the Multiple Sclerosis Functional Composite. Neurology 2003; 61:1367-1373.
Beck R W, Trobe J D, Moke P S, Optic Neuritis Study Group. High- and low-risk profiles for the development of multiple sclerosis within 10 years after optic neuritis: experience of the optic neuritis treatment trial. Arch Ophthal 2003; 121:944-949.
Canfield S M, Morrison S L. The binding affinity of human IgG for its high affinity Fc receptor is determined by multiple amino acids in the CH2 domain and is modulated by the hinge region. J Exp Med 1991; 173:1483-1491.
Cheema et al. Arthritis Rheum 2001; 44(6): 1313-1319.
Chen L, Gordon L K. Ocular manifestations of multiple sclerosis. Curr Opin Opthalmol 2005; 16:315-320.
Costello F, Coupland S, Hodge W, Lorello G R, Koroluk J, Pan Y I et al. Quantifying axonal loss after optic neuritis with optical coherence tomography. Ann Neurol 2006; 59:963-969.
Costello F. Optic neuritis: the role of disease-modifying therapy in this clinically isolated syndrome. Curr Treat Options Neurol 2007; 9:48-54.
Cross A, et al. Effects of rituximab on serum and spinal fluid B cells and antibodies in multiple sclerosis. ECTRIMS. 2005. Abstract.
Devereux J et al, Nucleic Acids Res, 12, 387-395, 1984.
Do R K G, Hatada E, Lee H, Tourigny M R, Hilbert D, Chen-Kiang S. Attenuation of apoptosis underlies B lymphocyte stimulator enhancement of humoral immune response. J Exp Med 2000; 192(7):953-964.
Ferguson B, Matyszak M K, Esiri M M, Perry V H. Axonal damage in acute multiple sclerosis lesions. Brain 1997; 120:393-399.
Fisher S A, Jacobs D A, Markowitz C E, Galetta S L, Volpe N J, Nano-Schiavi M L et al. Relation of visual function to retinal nerve fiber layer thickness in multiple sclerosis. Opthalmology 2006; 113:324-332.
Frohman E, Costello F, Zivadinov R, Stuve O, Conger A, Winslow H et al. Optical coherence tomography in multiple sclerosis. Lancet Neurol 2006; 5:853-863.

Fu L, Matthews P M, De Stefano N, Worsley K J, Narayanan S, Francis G S et al. Imaging axonal damage of normal appearing white matter in multiple sclerosis. Brain 1998; 121:103-113.
Gilbert M E, Sergott R C. New directions in optic neuritis and multiple sclerosis. Curr Neurol Neurosci Rep 2007; 7:259-264.
Grantham et al., Science, Vol. 185, pp. 862-864 (1974).
Groom et al. J Clin Invest 2002; 109(I):59-68; Mariette X, Ann Rheum Dis 2003; 62(2):168-171.
Gross et al. Nature 2000; 404:995-999;
Haubold K, Owens G P, Kaur P, Ritchie A M, Gilden D H, Bennett J L. B-lymphocyte and plasma cell clonal expansion in monosymptomatic optic neuritis cerebrospinal fluid. Ann Neurol 2004; 56:97-107.
Hee M R, Izatt J A, Swanson E A, Huang D, Schuman J S, Lin C P et al. Optical coherence tomography of the human retina. Arch Ophthal 1995; 113:325-332.
Hirtz D, Thurman D J, Gwinn-Hardy K, Mohamed M, Chaudhuri A R, Zalutsky R. How common are the “common” neurologic disorders? Neurology 2007; 68:326-337.
Huang D, Swanson E A, Lin C P, Schuman J S, Stinson W G, Chang W et al. Optical coherence tomography. Science 1991; 254:1178-1181.
Kabat E A, Glusman M, Knaub V. Quantitative estimation of the albumin and gamma globulin in normal and pathologic cerebrospinal fluid by immunochemical methods. Am J Med 1948; 4:653-662.
Kabat, E. A., Wu, T. T., Perry, H. M., Gottesman, K. S., and Foeller, C. (1991), Sequences of Proteins of Immunological Interest, 5th Ed., National Institutes of Health, Bethesda, Md.
Klawiter E C, Cross A H. B cells: no longer the nondominant arm of multiple sclerosis. Curr Neurol Neurosci Rep 2007; 7:231-238.
Mackay et al. J Exp Me 1999; 190(11); 1697-1710.
Marsters S A, Yan M, Pith R M, Haas P E, Dixit V M, Ashkenazi A. Interaction of the TNF homologues BLyS and APRIL with the TNF receptor homologues BCMA and TACI. Curr Biol 2000; 10(13):785-788.
Moore et al. Science 1999; 285(5425): 260-263.
Ogden T E. Nerve fiber layer of the primate retina: thickness and glial content. Vision Res 1983; 23:581-587.
Optic Neuritis Study Group. The clinical profile of optic neuritis: Experience of the Optic Neuritis Treatment Trial. Arch Opthalmol 1991; 109(12):1673-1678.
Pearson, Methods Enzymol. 1990; 183:63-98.
Polman C H, Reingold S C, Edan G, Filippi M, Hartung H-P, Kappos L et al. Diagnostic criteria for multiple sclerosis. 2005 revisions to the “McDonald Criteria”. Ann Neurol 2005; 58:840-846.
Rodriguez M, Siva A, Cross S A, O'Brien P C, Kurland L T. Optic neuritis: A population-based study in Olmsted County, Minnesota. Neurology 1995; 45:244-250.
Roschke V, Sosnovtseva S, Ward C D, Hong J S, Smith R, Albert V et al. BLyS and APRIL form biologically active heterotrimers that are expressed in patients with systemic immune-based rheumatic diseases. J Immunol 2002; 169:4314-4321.
Rudick et al., Neurology 2001; 56:1324-1330.
Schneider P, Mackay F, Steiner V, Hofmann K, Bodmer J-L, Holler N. BAFF, a novel ligand of the tumor necrosis factor family, stimulates B cell growth. J Exp Med 1999; 189(11):1747-1756.
Schneider et al. J Exp Med 1999; 189(11): 1747-1756; Do et al. J Exp Med 2000; 192(7):953-964.
Sergott R C. Optical coherence tomography: measuring in-vivo axonal survival and neuroprotection in multiple sclerosis and optic neuritis. Curr Opin Opthalmol 2005; 16:346-350.
Shields R L. et al., 2001. High resolution mapping of the binding site on human IgG1 for Fc gamma RI, Fc gamma RII, Fc gamma RIII, and FcRn and design of IgG1 variants with improved binding to the Fc gamma R. J Biol. Chem. 276(9):6591-604.
Soderstrom M, Link H, Xu Z, Fredriksson S. Optic neuritis and multiple sclerosis: Anti-MBP and anti-MBP peptide antibody-secreting cells are accumulated in CSF. Neurology 1993; 43:1215-1222.
Sorensen T L, Frederiksen J L, Bronnum-Hansen H, Petersen H C. Optic neuritis as onset manifestation of multiple sclerosis. A nationwide, long-term survey. Neurology 1999; 53:473-478.
Tao M-H, Smith R I F, Morrison S L. Structural features of human immunoglobulin G that determine isotype-specific differences in complement activation. J Exp Med 1993; 178:661-667.
Thompson J S, Bixler S A, Qian F, Vora K, Scott M L, Cachero T G. BAFF-R, a newly identified TNF receptor that specifically interacts with BAFF. Science 2001; 293:2108-2111.
Trip S A, Schlottmann P G, Jones S J, Altmann D R, Garway-Heath D F, Thompson A J et al. Retinal nerve fiber layer axonal loss and visual dysfunction in optic neuritis. Ann Neurol 2005; 58:383-391.
Trope and Britton, British Journal of Ophtalmology 1987, 71: 489-493.

1. A TACI-immunoglobulin (TACI-Ig) fusion protein comprising
a) the TACI extracellular domain or a fragment or variant thereof which binds to BLyS and/or APRIL; and
b) an immunoglobulin-constant domain for treatment of optic neuritis as clinically isolated syndrome.

2. The TACI-Ig fusion protein according to claim 1 for prevention of the conversion of optic neuritis to relapsing multiple sclerosis or clinically definite multiple sclerosis.

3. The TACI-Ig fusion protein according to claim 1 for prevention of recurrence of optic neuritis.

4. The TACI-Ig fusion protein according to claim 1 for treatment of symptomatic unilateral optic neuritis.

5. The TACI-Ig fusion protein according to claim 1 for treatment of monofocal optic neuritis.

6. The TACI-Ig fusion protein according to claim 1, wherein said TACI extracellular domain comprises the sequence of SEQ ID NO: 1 or a variant thereof being at least 90% or 95% or 99% identical to SEQ ID NO: 1, or a variant thereof comprising less than 20 conservative amino acids substitutions, the variant binding to BLyS and/or APRIL.

7. The TACI-Ig fusion protein according to claim 1, wherein said fragment comprises amino acid residues 34 to 66 and/or amino acid residues 71 to 104 of SEQ ID NO: 1.

8. The TACI-Ig fusion protein according to claim 1, wherein said fragment comprises amino acid residues 30 to 110 of SEQ ID NO: 1, or a variant thereof being at least 90% identical thereto or having less than 10 conservative amino acid substitutions, the variant binding to BLyS and/or APRIL.

9. The TACI-Ig fusion protein according to claim 1, wherein said immunoglobulin-constant domain is a human IgG1 constant domain.

10. The TACI-Ig fusion protein according to claim 9, wherein the human IgG1 constant domain has been modified for reduced complement dependent cytotoxicity (CDC) and/or antibody dependent cellular cytotoxicity (ADCC).

11. The TACI-Ig fusion protein according to claim 1, wherein said human immunoglobulin-constant domain has the sequence of SEQ ID NO: 2 or a variant thereof comprising less than 20 conservative amino acid substitutions.

12. The TACI-Ig fusion protein according to claim 1, comprising a sequence of SEQ ID NO: 3, or a variant thereof being at least 90% identical thereto or having less than 30 conservative amino acid substitutions, the variant binding to BLyS and/or APRIL.

13. The TACI-Ig fusion protein according to claim 1, formulated for administration in an amount of 150 mg per patient per day.

14. The TACI-Ig fusion protein according to claim 1, formulated for administration twice a week or weekly.

15. The TACI-Ig fusion protein according to claim 1, formulated for administration twice a week during a loading period and formulated for administration once a week during a maintenance period.

16. The TACI-Ig fusion protein according to claim 15, wherein the loading period is up to one month and the maintenance period is at least 8 months.

17. The TACI-Ig fusion protein according to claim 1, formulated for subcutaneous administration.

18. The TACI-Ig fusion protein according to claim 1, formulated in a sodium acetate buffer at pH 5 comprising trehalose.
